(19) United States
(12) Patent Application Publication
(10) Pub. No.: US 2008/0227863 A1
(43) Pub. Date: Sep. 18, 2008

(54) Organic Compounds

(75) Inventors: Pierre Ducray, Village-Neuf (FR); Jorg Fruchtel, Lorrach (DE); Noelle Gauvry, Kembs-Loechle (FR); Sandra Schorderet Weber, Neuchatel (CH)

Correspondence Address: Novartis Animal Health US Inc., 3200 Northline Avenue, Suite 300, Greensboro, NC, 27408, US

(73) Assignee: Novartis AG

(21) Appl. No.: 11/996,895

(22) PCT Filed: Jul. 24, 2006
(86) PCT No.: PCT/EP06/07259

§ 371 Date: Jan. 25, 2008

(30) Foreign Application Priority Data
Jul. 25, 2005 (EP) 05016071.2

(51) Int. Cl. A61K 31/166; C07C 233/71; A01N 37/18; A01P 15/00; A61P 33/00

(52) U.S. Cl. 514/617; 564/185

(57) ABSTRACT

The invention relates to compounds of the general formula

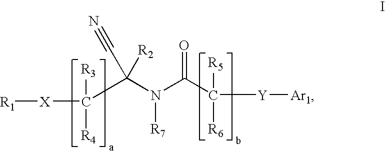

wherein R₁, R₂, R₃, R₄, R₅, R₆, R₇, Ar₁, X, Y, a and b are as defined in claim 1, and, if appropriate, enantiomers thereof. The active compounds have advantageous pesticidal properties. They are suitable, in particular, for the control of parasites in and on warm-blooded animals.

The present invention relates to new amidonitrile compounds of formula

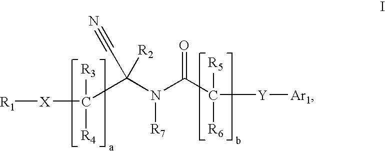

wherein
R₁ is hydrogen, C₁-C₆-alkyl; aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₆R₉, COOR₈, unsubstituted or one- to five-fold substituted aryl, unsubstituted or one- to five-fold substituted aryloxy, unsubstituted or one- to five-fold substituted arylthio, unsubstituted or one- to five-fold substituted hetaryl or unsubstituted or one- to five-fold substituted hetaryloxy, the substituents selected from the group consisting of halogen, cyano, NO₂, hydroxy, C₁-C₆-alkyl, halo-C₁-C₆-alkyl, C₁-C₆-alkoxy, halo-C₁-C₆-alkoxy, NH₂, C₁-C₆-alkylamino, di-C₁-C₆-alkylamino, C₁-C₆-alkylthio, COR₈, CONR₆R₉ and COOR₈;
R₂ is cyano, CONR₈R₉ or COOR₈;
R₃, R₄, R₅ and R₆, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
R₇ is hydrogen; C₁-C₆-alkyl, C₂-C₆-alkenyl, C₂-C₆-alkynyl or C₃-C₆-cycloalkyl each optionally substituted with one or more substituents selected from the group consisting of halogen, cyano, hydroxy and C₁-C₆-alkoxy; or C₁-C₆-alkylcarbonyl or C₁-C₆-alkoxycarbonyl; either R₈ and R₉, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
or, together with the nitrogen atom to which they are attached, form a ring of 3 to 6 atoms, optionally including one additional atom of nitrogen, sulfur or oxygen, optionally substituted with 1 to 4 substituents selected form the group consisting of halogen, CN, NO₂, hydroxy, C₁-C₆-alkyl and C₁-C₆-alkoxy;
Ar₁ is aryl or hetaryl each optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉ and COOR₈;
X and Y are independently of one another a direct bond or oxygen; and
a and b, independently of one another, are 0, 1 or 2;
their preparation and use in the control of endo- and ectoparasites, especially helminths, in and on warm-blooded productive livestock and domestic animals and plants, and furthermore pesticides containing at least one of these compounds.

Substituted amidonitrile compounds having pesticidal activity are described for example in EP-0.953.565 A2. However, the active ingredients specifically disclosed therein cannot always fulfil the requirements regarding potency and activity spectrum. There is therefore a need for active ingredients with improved pesticidal properties. It has now been found that the amidonitrile compounds of formula I have excellent pesticidal properties, especially against endo- and ecto-parasites in and on productive livestock and domestic animals and plants.

The general terms used hereinbefore and hereinafter have the following meanings, unless defined otherwise.

Alkyl—as a group per se and as structural element of other groups and compounds such as halogen-alkyl, alkylamino, alkoxy, alkylthio, alkylsulfinyl and alkylsulfonyl—is, in each case with due consideration of the specific number of carbon atoms in the group or compound in question, either straight-chained, i.e. methyl, ethyl, propyl, butyl, pentyl, hexyl, heptyl or octyl, or branched, e.g. isopropyl, isobutyl, sec.-butyl, tert.-butyl, isopentyl, neopentyl or isohexyl.

Cycloalkyl—as a group per se and as structural element of other groups and compounds such as halocycloalkyl, cycloalkoxy and cycloalkylthio, —is, in each case with due consideration of the specific number of carbon atoms in the group or compound in question, cyclopropyl, cyclobutyl, cyclopentyl, cyclohexyl, cycloheptyl or cyclooctyl.

Alkenyl—as a group per se and as structural element of other groups and compounds—is, in each case with due consideration of the specific number of carbon atoms in the group or compound in question and of the conjugated or isolated double bonds—either straight-chained, e.g. allyl, 2-butenyl, 3-pentenyl, 1-hexenyl, 1-heptenyl, 1,3-hexadienyl or 1,3-octadienyl, or branched, e.g. isopropenyl, isobutenyl, isoprenyl, tert.-pentenyl, isohexenyl, isoheptenyl or isooctenyl.

Alkynyl—as a group per se and as structural element of other groups and compounds—is, in each case with due consideration of the specific number of carbon atoms in the group or compound in question and of the conjugated or isolated double bonds—either straight-chained, e.g. propargyl, 2-butinyl, 3-pentinyl, 1-hexinyl, 1-heptinyl, 3-hexen-1-inyl or 1,5-heptadien-3-inyl, or branched, e.g. 3-methylbut-1-inyl, 4-ethylpent-1-inyl, 4-methylhex-2-inyl or 2-methylhept-3-inyl.

Aryl is, for example, phenyl or naphthyl, in particular phenyl. R₁ as phenyl is, for example, phenyl which is unsubstituted or substituted with one or more substituents as mentioned before, preferably phenyl which is unsubstituted or substituted by 1 to 3 and in particular 1 or 2 substituents. Ar₁ as phenyl is unsubstituted or one- to fivefold substituted phenyl, preferably unsubstituted or one- to twofold substituted phenyl and in particular unsubstituted or mono-substituted phenyl, wherein the substituents are in each case as mentioned before.

Hetaryl is, for example, pyridyl, pyrimidyl, s-triazinyl, 1,2,4-triazinyl, thienyl, furanyl, pyrryl, pyrazolyl, imidazolyl, thiazolyl, triazolyl, oxazolyl, thiadiazolyl, oxadiazolyl, benzothienyl, benzofuranyl, benzothiazolyl, indolyl or indazolyl, preferably pyridyl, pyrimidyl, pyrryl, imidazolyl or furanyl, in particular pyridyl or pyrimidyl.

As a rule, halogen signifies fluorine, chlorine, bromine or iodine. The same applies to halogen in combination with other significances, such as halogenalkyl.

Halogen-substituted carbon-containing groups and compounds may be partially halogenated or perhalogenated, whereby in the case of multiple halogenation, the halogen substituents may be identical or different. Examples of halogen-alkyl—as a group per se and as structural element of other groups and compounds such as halogen-alkoxy or halogen-alkylthio, —are methyl which is mono- to trisubstituted by fluorine, chlorine and/or bromine, such as CHF₂ or CF₃; ethyl which is mono- to pentasubstituted by fluorine, chlorine and/or bromine, such as CH₂CF₃, CF₂CF₃, CF₂CCl₃, CF₂CHCl₂, CF₂CHF₂, CF₂CFCl₂, CF₂CHBr₂, CF₂CHClF, CF₂CHBrF or CClFCHClF; propyl or isopropyl, mono- to heptasubstituted by fluorine, chlorine and/or bromine, such as CH₂CHBrCH₂Br, CF₂CHFCF₃, CH₂CF₂CF₃ or CH(CF₃)₂; butyl or one of its isomers, mono- to nonasubstituted by fluorine, chlorine and/or bromine, such as CF(CF₃)CHFCF₃ or CH₂(CF₂)₂CF₃; pentyl or one of its isomers substituted once to eleven times by fluorine, chlorine and/or bromine, such as CF(CF₃)(CHF)₂CF₃ or CH₂(CF₂)₃CF₃; and hexyl or one of its isomers substituted once to thirteen times by fluorine, chlorine and/or bromine, such as (CH₂)₄CHBrCH₂Br, CF₂(CHF)₄CF₃, CH₂(CF₂)₄CF₃ or C(CF₃)₂(CHF)₂CF₃.

Alkoxy groups preferably have a chain length of 1 to 6 carbon atoms. Alkoxy is for example methoxy, ethoxy, propoxy, isopropoxy, n-butoxy, isobutoxy, sec.-butoxy and tert.-butoxy, as well as the isomers pentyloxy and hexyloxy; preferably methoxy and ethoxy. Halogenalkoxy groups preferably have a chain length of 1 to 6 carbon atoms. Halogenalkoxy is e.g. fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy and 2,2,2-trichloroethoxy; preferably difluoromethoxy, 2-chloroethoxy and trifluoromethoxy.

Alkylthio groups preferably have a chain length of 1 to 6 carbon atoms. Alkylthio is, for example, methylthio, ethylthio, propylthio, isopropylthio, n-butylthio, isobutylthio, sec-butylthio or tert-butylthio, preferably methylthio and ethylthio.

Preferred embodiments within the context of the invention are:

1. a compound of the formula I, wherein R₁ is aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₂NR₈R₉, unsubstituted or one- to five-fold substituted aryl, unsubstituted or one- to five-fold substituted aryloxy, or unsubstituted or one- to five-fold substituted arylthio, the substituents selected from the group consisting of halogen, cyano, C₁-C₆-alkyl, halo-C₁-C₆-alkyl, C₁-C₆-alkoxy and halo-C₁-C₆-alkoxy;
preferably aryl optionally substituted with one or more substitutents selected from the group consisting of halogen, cyano, C₁-C₄-alkyl, C₂-C₄-alkenyl, halo-C₁-C₄-alkyl, halo-C₂-C₄-alkenyl, C₁-C₄-alkoxy, C₂-C₄-alkenyloxy, halo-C₁-C₄-alkoxy, halo-C₂-C₄-alkenyloxy, C₁-C₄-alkylthio, halo-C₁-C₄-alkylthio, unsubstituted or one- to five-fold substituted aryl, unsubstituted or one- to five-fold substituted aryloxy, or unsubstituted or one- to five-fold substituted arylthio, the substituents selected from the group consisting of halogen, cyano, C₁-C₆-alkyl, halo-C₁-C₆-alkyl, C₁-C₆-alkoxy and halo-C₁-C₆-alkoxy;
more preferably phenyl optionally substituted with one or more substitutents selected from the group consisting of halogen, cyano, C₁-C₂-alkyl, halo-C₁-C₂-alkyl, C₁-C₂-alkoxy, halo-C₁-C₂-alkoxy, C₁-C₂-alkylthio, halo-C₁-C₂-alkylthio, unsubstituted or one- to five-fold substituted phenoxy, or unsubstituted or one- to five-fold substituted arylthio, the substituents selected from the group consisting of halogen, cyano, C₁-C₄-alkyl, halo-C₁-C₄-alkyl, C₁-C₄-alkoxy and halo-C₁-C₄-alkoxy;
most preferably phenyl optionally substituted with one or more substitutents selected from the group consisting of halogen, cyano, methyl, halo-methyl, methoxy and unsubstituted or mono- or dihalogen substituted phenoxy;
2. a compound of the formula I, wherein R₂ is cyano or COOR₈; preferably cyano;
3. a compound of the formula I, wherein R₃, R₄, R₅ and R₆, independently of one another, are hydrogen or C₁-C₆-alkyl, each optionally substituted with one or more substituents selected from the group consisting of halogen;
preferably hydrogen or C₁-C₄-alkyl;
more preferably hydrogen;
4. a compound of the formula I, wherein R₇ is hydrogen or C₁-C₆-alkyl;
preferably hydrogen or methyl;
most preferably hydrogen;
5. a compound of the formula I, wherein R₈ and R₉, independently of one another, are hydrogen or C₁-C₆-alkyl, optionally substituted with one or more substituents selected from the group consisting of halogen, cyano, NO₂ and C₁-C₄-alkoxy;
preferably, independently of one another, hydrogen or C₁-C₄-alkyl;
more preferably C₁-C₄-alkyl;
most preferably C₁-C₂-alkyl;
6. a compound of the formula I, wherein Ar₁ is aryl optionally substituted with one or more substitutents selected from the group consisting of halogen, cyano, NO₂, C₁-C₆-alkyl, C₂-C₆-alkenyl, halo-C₁-C₆-alkyl, C₁-C₆-alkoxy, C₂-C₆-alkenyloxy, halo-C₁-C₆-alkoxy, halo-C₂-C₆-alkenyloxy, C₁-C₆-alkylthio, halo-C₁-C₆-alkylthio, C₁-C₆-alkylsulfonyl, halo-C₁-C₆-alkylsulfonyl, C₁-C₆-alkylamino and di-C₁-C₆-alkylamino;
preferably phenyl optionally substituted with one or more substitutents selected from the group consisting of halogen, cyano, NO₂, C₁-C₄-alkyl, halo-C₁-C₄-alkyl, C₁-C₄-alkoxy, halo-C₁-C₄-alkoxy, C₁-C₄-alkylthio and halo-C₁-C₄-alkylthio;
more preferably phenyl optionally substituted with one or more substitutents selected from the group consisting of halogen, cyano, NO₂, C₁-C₂-alkyl, halo-C₁-C₂-alkyl, C₁-C₂-alkoxy, halo-C₁-C₂-alkoxy, C₁-C₂-alkylthio and halo-C₁-C₂-alkylthio;
most preferably phenyl optionally substituted with one or more substitutents selected from the group consisting of halogen, cyano, NO₂, methyl, halo-methyl, methoxy, halo-methoxy, methylthio and halo-methylthio;
7. a compound of the formula I, wherein X is oxygen;
8. a compound of the formula I, wherein Y is a direct bond;
9. a compound of the formula I, wherein a and b, independently of one another, are 0 or 1; preferably a 1 and b 0;
10. a compound of the formula I, wherein R₁ is aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₂NR₈R₉, unsubstituted or one- to five-fold substituted aryl, unsubstituted or one- to five-fold substituted aryloxy, or unsubstituted or one- to five-fold substituted arylthio, the substituents selected from the group consisting of halogen, cyano, C₁-C₆-alkyl, halo-C₁-C₆-alkyl, C₁-C₆-alkoxy and halo-C₁-C₆-alkoxy;
R₂ is cyano or COOR₈;
R₃, R₄, R₅ and R₆, independently of one another, are hydrogen or C₁-C₆-alkyl, each optionally substituted with one or more substituents selected from the group consisting of halogen;
R₇ is hydrogen or C₁-C₆-alkyl;
R₈ is hydrogen or C₁-C₆-alkyl, optionally substituted with one or more substituents selected from the group consisting of halogen, cyano, NO₂ and C₁-C₄-alkoxy;
Ar₁ is aryl optionally substituted with one or more substitutents selected from the group consisting of halogen, cyano, NO₂, C₁-C₆-alkyl, C₂-C₆-alkenyl, halo-C₁-C₆-alkyl, C₁-C₆-alkoxy, C₂-C₆-alkenyloxy, halo-C₁-C₆-alkoxy, halo-C₂-C₆-alkenyloxy, C₁-C₆-alkylthio, halo-C₁-C₆-alkylthio, C₁-C₆-alkylsulfonyl, halo-C₁-C₆-alkylsulfonyl, C₁-C₆-alkylamino and di-C₁-C₆-alkylamino; and
a and b, independently of one another, are 0 or 1;
11. a compound of the formula I, wherein R₁ is aryl optionally substituted with one or more substitutents selected from the group consisting of halogen, cyano, C₁-C₄-alkyl, C₂-C₄-alkenyl, halo-C₁-C₄-alkyl, halo-C₂-C₄-alkenyl, C₁-C₄-alkoxy, C₂-C₄-alkenyloxy, halo-C₁-C₄-alkoxy, halo-C₂-C₄-alkenyloxy, C₁-C₄-alkylthio, halo-C₁-C₄-alkylthio, unsubstituted or one- to five-fold substituted aryl, unsubstituted or one- to five-fold substituted aryloxy, or unsubstituted or one- to five-fold substituted arylthio, the substituents selected from the group consisting of halogen, cyano, C₁-C₆-alkyl, halo-C₁-C₆-alkyl, C₁-C₆-alkoxy and halo-C₁-C₆-alkoxy;
R₂ is cyano;
R₃, R₄, R₅ and R₆, independently of one another, are hydrogen or C₁-C₆-alkyl;
R₇ is hydrogen or methyl;
Ar₁ is phenyl optionally substituted with one or more substitutents selected from the group consisting of halogen, cyano, NO₂, C₁-C₄-alkyl, halo-C₁-C₄-alkyl, C₁-C₄-alkoxy, halo-C₁-C₄-alkoxy, C₁-C₄-alkylthio and halo-C₁-C₄-alkylthio;
a is 1 and b is 0;
12. a compound of the formula I, wherein R₁ is phenyl optionally substituted with one or more substitutents selected from the group consisting of halogen, cyano, C₁-C₂-alkyl, halo-C₁-C₂-alkyl, C₁-C₂-alkoxy, halo-C₁-C₂-alkoxy, C₁-C₂-alkylthio, halo-C₁-C₂-alkylthio, unsubstituted or one- to five-fold substituted phenoxy, or unsubstituted or one- to five-fold substituted arylthio, the substituents selected from the group consisting of halogen, cyano, C₁-C₄-alkyl, halo-C₁-C₄-alkyl, C₁-C₄-alkoxy and halo-C₁-C₄-alkoxy;
R₂ is cyano;
R₃, R₄, R₅, R₆ and R₇ are hydrogen;
Ar₁ is phenyl optionally substituted with one or more substitutents selected from the group consisting of halogen, cyano, NO₂, C₁-C₂-alkyl, halo-C₁-C₂-alkyl, C₁-C₂-alkoxy, halo-C₁-C₂-alkoxy, C₁-C₂-alkylthio and halo-C₁-C₂-alkylthio;
X is oxygen;
Y is a direct bond;
a is 1 and b is 0;
13. a compound of the formula I, wherein R₁ is phenyl optionally substituted with one or more substitutents selected from the group consisting of halogen, cyano, methyl, halo-methyl, methoxy or mono- or dihalogen substituted phenoxy;
R₂ is cyano;
R₃, R₄, R₅, R₆ and R₇ are hydrogen;
Ar₁ is phenyl optionally substituted with one or more substitutents selected from the group consisting of halogen, cyano, NO₂, methyl, halo-methyl, methoxy, halo-methoxy, methylthio and halo-methylthio;
X is oxygen;
Y is a direct bond;
a is 1 and b is 0;
14. a compound of formula

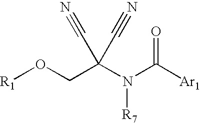

wherein for R₁, R₇ and Ar₁ the above given meanings and preferences apply.

Particularly preferred compounds of the formula I for the purposes of the invention are those listed in table 1 and very particularly preferably the compounds of the formula I mentioned in the synthesis examples.

A further subject of the invention is the process for the preparation of the compounds of the formula I, in each case in free form or in salt form, characterized in that a compound of the formula

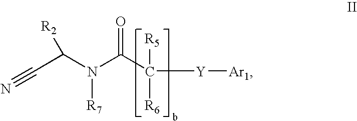

in which R₂, R₅, R₆, R₇, Ar₁, Y and b are as defined for the formula I, is reacted with a compound of the formula

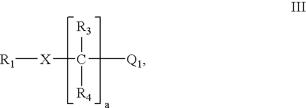

in which R₁, R₃, R₄, X and a are as defined for the formula I and Q₁ is a leaving group, if appropriate in the presence of a basic catalyst. If desired, a compound of the formula I, in each case in free form or in salt form, obtainable according to the above described process or in another manner, may be converted into another compound of the formula I, a mixture of enantiomers obtainable according to the process is separated and the desired enantiomer is isolated and/or a free compound of the formula I obtainable according to the process is converted into a salt or a salt of a compound of the formula I obtainable according to the process is converted into the free compound of the formula I or into another salt.

The starting materials of the above process are known or may be obtained according to processes known per se.

The reactants can be reacted with one another as such, that is without addition of a solvent or diluent, e.g. in the melt. Usually, however, the addition of an inert solvent or diluent or of a mixture thereof is advantageous. Examples of such solvents or diluents which may be mentioned are: aromatic, aliphatic and alicyclic hydrocarbons and halogenated hydrocarbons, such as benzene, toluene, xylene, mesitylene, tetralin, chlorobenzene, dichlorobenzene, bromobenzene, petroleum ether, hexane, cyclohexane, dichloromethane, trichloromethane, tetrachloromethane, dichloroethane, trichloroethene or tetrachloroethene; ethers, such as diethyl ether, dipropyl ether, diisopropyl ether, dibutyl ether, tert-butyl methyl ether, ethylene glycol monomethyl ether, ethylene glycol monoethyl ether, ethylene glycol dimethyl ether, dimethoxydiethyl ether, tetrahydrofuran or dioxane; ketones, such as acetone, methyl ethyl ketone or methyl isobutyl ketone; amides, such as N,N-dimethylformamide, N,N-diethyl-formamide, N,N-dimethylacetamide, N-methylpyrrolidone or hexamethylphosphoramide; nitriles, such as acetonitrile or propionitrile; sulphoxides, such as dimethyl sulphoxide; or amines, such as triethylamine, pyridine or 4-dimethylaminopyridine.

Preferred solvents are amides, in particular N-methylpyrrolidone.

Preferred leaving groups Q₁ are halogens, in particular chlorine.

Suitable bases for facilitating the reaction are, for example, alkali metal or alkaline earth metal hydroxides, hydrides, amides, alkoxides, acetates, carbonates, dialkylamides or alkylsilylamides, alkylamines, alkylenediamines, free or N-alkylated, unsaturated or saturated, cycloalkylamines, basic heterocycles, ammonium hydroxides, and carbocyclic amines. Examples which may be mentioned are sodium hydroxide, hydride, amide, methoxide, acetate, carbonate, potassium t-butoxide, hydroxide, carbonate, hydride, lithium diisopropyl-amide, potassium bis(trimethylsilyl)amide, calcium hydride, triethylamine, diisopropylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethyl-ammonium hydroxide, and 1,5-diazabicyclo[5.4.0]undec-5-ene (DBU).

Preferred bases are metal hydrides, in particular sodium hydride.

The reaction is advantageously carried out in a temperature range from approximately 0° C. to approximately +80° C., preferably from approximately 10° C. to approximately +30° C.

A further subject of the invention is the process for the preparation of the compounds of the formula II, in each case in free form or in salt form, for example characterized in that a compound of the formula

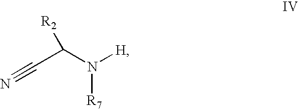

in which R₂ and R₇ are as defined for the formula I, is reacted with a compound of formula

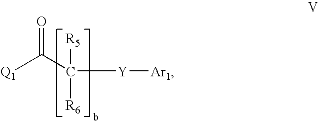

in which R₅, R₆, Ar₁, Y and b are as defined for the formula I and Q₁ is a leaving group. If desired, a compound of the formula II, in each case in free form or in salt form, obtainable according to the invention or in another manner, may be converted into another compound of the formula II, a mixture of enantiomers obtainable according to the process is separated and the desired enantiomer is isolated and/or a free compound of the formula II obtainable according to the process is converted into a salt or a salt of a compound of the formula II obtainable according to the process is converted into the free compound of the formula II or into another salt.

The reactants, which are known or may be obtained according to processes known per se, can be reacted with one another as such, that is without addition of a solvent or diluent, e.g. in the melt. Usually, however, the addition of an inert solvent or diluent or of a mixture thereof is advantageous. Examples of such solvents or diluents which may be mentioned are: aromatic, aliphatic and alicyclic hydrocarbons and halogenated hydrocarbons, such as benzene, toluene, xylene, mesitylene, tetralin, chlorobenzene, dichlorobenzene, bromobenzene, petroleum ether, hexane, cyclohexane, dichloromethane, trichloromethane, tetrachloromethane, dichloroethane, trichloroethene or tetrachloroethene; ethers, such as diethyl ether, dipropyl ether, diisopropyl ether, dibutyl ether, tert-butyl methyl ether, ethylene glycol monomethyl ether, ethylene glycol monoethyl ether, ethylene glycol dimethyl ether, dimethoxydiethyl ether, tetrahydrofuran or dioxane; ketones, such as acetone, methyl ethyl ketone or methyl isobutyl ketone; amides, such as N,N-dimethylformamide, N,N-diethyl-formamide, N,N-dimethylacetamide, N-methylpyrrolidone or hexamethylphosphoramide; nitriles, such as acetonitrile or propionitrile; sulphoxides, such as dimethyl sulphoxide; or amines, such as triethylamine, pyridine or 4-dimethylaminopyridine.

Preferred solvents are amines; particularly preferred is pyridine.

The reaction is advantageously carried out in a temperature range from approximately −10° C. to approximately +60° C., preferably from approximately 0° C. to approximately +10° C.

Compounds of formula I obtainable in accordance with the process or by another method can be converted in a manner known per se into different compounds of formula I by replacing one or more substituents of the starting compound of formula I by (an)other substituent(s) according to the invention in customary manner.

Depending upon the reaction conditions and starting materials selected as suitable in each case, it is possible in a reaction step to replace only one substituent by another substituent according to the invention or it is possible in the same reaction step to replace a plurality of substituents by other substituents according to the invention.

Salts of compounds of formula I can be prepared in a manner known per se. For example, acid addition salts of compounds of formula I are obtained by treatment with a suitable acid or a suitable ion exchange reagent and salts with bases are obtained by treatment with a suitable base or a suitable ion exchange reagent.

Salts of compounds of formula I can be converted in customary manner into the free compounds of formula I; acid addition salts can be converted, for example, by treatment with a suitable basic medium or a suitable ion exchange reagent and salts with bases, for example, by treatment with a suitable acid or a suitable ion exchange reagent.

Salts of compounds of formula I can be converted into different salts of compounds of formula I in a manner known per se; for example acid addition salts can be converted into different acid addition salts, for example by treatment of a salt of an inorganic acid, such as a hydrochloride, with a suitable metal salt, such as a sodium, barium or silver salt, of an acid, for example with silver acetate, in a suitable solvent in which an inorganic salt being formed, for example silver chloride, is insoluble and is therefore precipitated out from the reaction mixture.

Depending upon the procedure and/or the reaction conditions, the compounds of formula I having salt-forming properties can be obtained in free form or in the form of salts.

The compounds of formula I can also be obtained in the form of their hydrates and/or can include other solvents, for example any solvent that may have been used for the crystallisation of compounds in solid form.

The compounds of formulae I and II may be in the form of one of the possible isomers or in the form of a mixture thereof, for example depending upon the number of asymmetric carbon atoms and the absolute and relative configuration thereof, in the form of pure isomers, such as antipodes and/or diastereoisomers, or in the form of mixtures of isomers, such as mixtures of enantiomers, for example racemates, mixtures of diastereoisomers or mixtures of racemates; the invention relates both to the pure isomers and to all possible mixtures of isomers and this is to be understood accordingly hereinbefore and hereinafter, even when stereochemical details are not specifically mentioned in each case.

Mixtures of diastereoisomers and mixtures of racemates of compounds of formulae I and II obtainable in accordance with the process—depending upon the starting materials and procedures chosen—or by other means can be separated into the pure diastereoisomers or racemates in known manner on the basis of the physico-chemical differences between the constituents, for example by fractional crystallisation, distillation and/or chromatography.

Mixtures of enantiomers or racemates so obtainable can be separated into the optical antipodes by known methods, for example by recrystallisation from an optically active solvent, by chromatography on chiral adsorbents, for example high-pressure liquid chromatography (HPLC) on acetyl cellulose, with the aid of suitable microorganisms, by cleavage with specific immobilised enzymes, or via the formation of inclusion compounds, for example using chiral crown ethers, in which case only one enantiomer is complexed.

Pure diastereoisomers and enantiomers can be obtained not only by separation of corresponding mixtures of isomers but also, according to the invention, by generally known methods of diastereoselective or enantioselective synthesis, for example by carrying out the process according to the invention with starting materials that have appropriate stereo-chemistry.

It is advantageous to isolate or synthesise whichever isomer, for example enantiomer, or mixture of isomers, for example mixture of enantiomers, is biologically more active, insofar as the individual components have different biological activity.

In the process of the present invention there are preferably used those starting materials and intermediates which result in the compounds of formula I described at the beginning as being especially valuable.

The invention especially relates to the preparation process described in the examples.

The invention relates also to the novel starting materials and intermediates that are used according to the invention in the preparation of compounds of formula I, to their use and to processes for the preparation thereof.

In the area of pest control, the compounds of formula I according to the invention are active ingredients exhibiting valuable preventive and/or curative activity with a very advantageous biocidal spectrum, even at low rates of concentration, while being well tolerated by warm-blooded organisms, fish and plants. The compounds are especially suitable for use in the area of controlling endo- and ecto-parasites of animals and plant-destructive fungi, insects, acari and nematodes. The active ingredients according to the invention are effective against all or individual development stages of normally sensitive animal pests, but also of resistant animal pests, such as insects and representatives of the order Acarina and Helminths like nematodes or trematodes. The pesticidal activity of the active ingredients according to the invention may manifest itself directly, i.e. in the mortality of the pests, which occurs immediately or only after some time, for example during moulting, or indirectly, for example in reduced oviposition and/or hatching rate, good activity corresponding to a mortality of at least 50 to 60%. The compounds of formula I are especially distinguished by an unusually long duration of action.

Said pests of plants include those mentioned in European Patent Application EP-A-736 252, page 5, line 55 to page 6, line 55. The pests mentioned therein are therefore included by reference in the subject matter of the present invention.

Compounds of formula I can be used for the control of phytopathogenic fungi such as Alternaria solani, Botrytis cinerea, Cercospora beticola, Cladosporium herbarum, Corticium rolfsi, Erysiphe graminis, Helminthosporium tritici repentis, Lepfosphaeria nodorum, Micronectriella nivalis, Monilinia fructigena, Mycosphaerella ligulicola, Mycosphaerella pinodes, Pyricularia oryzae, Rhizotonia solani, Scierotinia sclerotiorum, Uncinula necator and Venturia inaequalis.

The compounds of formula I can also be used against pests affecting hygiene, especially of the order Diptera with the families Sarcophagidae, Muscidae, Anophilidae and Culicidae; and of the orders Orthoptera, Dictyoptera (e.g. the family Blattidae such as Blattella germanica, Blatta orientalis, Periplaneta americana) and Hymenoptera (e.g. the family Formicidae and Vespidae).

The compounds of formula I also have long-lasting activity in the case of mites and insects that are parasites of plants. In the case of spider mites of the order Acarina, they are effective against eggs, nymphs and adults of Tetranychidae (Tetranychus spp. and Panonychus spp.).

They possess a high degree of activity in sucking insects of the order Homoptera, especially against pests of the families Aphididae, Delphacidae, Cicadellidae, Psyllidae, Loccidae, Diaspididae and Eriophydidae (e.g. rust mite on citrus fruit); of the orders Hemiptera, Heteroptera and Thysanoptera, and in phytophagous insects of the orders Lepidoptera, Coleoptera, Diptera and Orthoptera.

They are also suitable as a soil insecticide against pests in the soil.

The compounds according to the invention can be used to control, i.e. to inhibit or destroy, pests of the mentioned type occurring especially on plants, more especially on useful plants and ornamentals in agriculture, in horticulture and in forestry, or on parts of such plants, such as the fruits, blossoms, leaves, stems, tubers or roots, while in some cases parts of plants that grow later are still protected against those pests.

The compounds of formula I are therefore effective against all development stages of sucking and phytophagous insects on crops such as cereals, e.g. wheat, barley, rye, oats, rice, maize and sorghum; beet, such as sugar beet and fodder beet; fruit, e.g. pomes, stone fruit and soft fruit, such as apples, pears, plums, peaches, almonds, cherries and berries, e.g. strawberries, raspberries and blackberries; leguminous plants, such as beans, lentils, peas and soybeans; oil plants, such as rape, mustard, poppy, sesame, olives, sunflowers, coconut, castor oil, cocoa and groundnuts; cucurbitaceae, such as marrows, cucumbers and melons; fibre plants, such as cotton, flax, hemp and jute; citrus fruits, such as oranges, lemons, grapefruit and mandarins; vegetables, such as spinach, lettuce, asparagus, cabbages, carrots and onions; Solanaceae such as tomatoes, potatoes, aubergines, capsicum, tobacco and paprika; Lauraceae such as avocado, cinnamon and camphor; and, nuts, coffee, sugar cane, tea, pepper, vines, hops, bananas, natural rubber plants and ornamentals.

The compounds of formula I are also effective against plant-nematodes of the species Meloidogyne, Heterodera, Pratylenchus, Ditylenchus, Radopholus, Rizoglyphus and others.

In the context of the present invention, ectoparasites occurring as parasites on warm-blooded organisms are understood to mean especially insects, mites and ticks. Included are insects of the orders: Lepidoptera, Coleoptera, Homoptera, Heteroptera, Diptera, Thysanoptera, Orthoptera, Anoplura, Siphonaptera, Mallophaga, Thysanura, Isoptera, Psocoptera and Hymenoptera. Special mention may be made, however, of ectoparasites that trouble humans and animals and transmit pathogens, for example flies, such as Musca domestica, Musca vetustissima, Musca autumnalis, Fannia canicularis, Sarcophaga camaria, Lucilia cuprina, Hypoderma bovis, Hypoderma lineatum, Chrysomyia chloropyga, Dermatobia hominis, Cochliomyia hominivorax, Gasterophilus intestinalis, Oestrus ovis, biting flies such as Stomoxys calcitrans, Haematobia irritans irritans, Haematobia initans exigua, horse-flies (Tabanids) with the subfamilies of Tabanidae, such as Haematopota spp. (e.g. Haematopota pluvialis) and Tabanus spp. (e.g. Tabanus nigrovittatus) and Chrysopsinae, such as Chrysops spp. (e.g. Chrysops caecutiens); Hippoboscids, such as Melophagus ovinus (sheep ked); tsetse flies, such as Glossinia spp.; other biting insects like midges, such as Ceratopogonidae (biting midges), Simuliidae (Blackflies), Psychodidae (Sandflies); but also blood-sucking insects, for example mosquitoes, such as Anopheles spp, Aedes spp and Culex spp, fleas, such as Ctenocephalides felis and Ctenocephalides canis (cat and dog fleas), Xenopsylla cheopis, Pulex irritans, Ceratophyllius gallinae, Dermatophilus penetrans, blood-sucking lice (Anoplura), such as Linognathus spp, Haematopinus spp, Solenopotes spp, Pediculus humanis; but also chewing lice (Mallophaga), such as Bovicola (Damalinia) ovis, Bovicola (Damalinia)bovis and other Bovicola spp. Ectoparasites also include members of the order Acarina, such as mites (e.g. Chorioptes bovis, Cheyletiella spp., Dermanyssus gallinae, Demodex canis, Sarcoptes scabiei, Psoroptes ovis and Psorergates spp. and ticks. Known representatives of ticks are, for example, Boophilus, Amblyomma, Anocentor, Dermacentor, Haemaphysalis, Hyalomma, Ixodes, Rhipicentor, Margaropus, Rhipicephalus, Argas, Otobius and Ornithodoros and the like, which preferably infest warm-blooded animals including farm animals, such as cattle, horses, pigs, sheep and goats, poultry such as chickens, turkeys, guineafowls and geese, fur-bearing animals such as mink, foxes, chinchillas, rabbits and the like, as well as domestic animals such as cats and dogs, but also humans.

In addition, the compounds of formula I are especially effective against helminths, among which the endoparasitic nematodes and trematodes may be the cause of serious diseases of mammals and poultry, for example of sheep, pigs, goats, cattle, horses, donkeys, dogs, cats, guinea-pigs and ornamental birds. Typical nematodes in that indication are: Haemonchus, Trichostrongylus, Teladosargia, Ostertagia, Nematodirus, Cooperia, Ascaris, Bunostonum, Oesophagostonum, Chabertia, Trichuris, Strongylus, Trichonema, Dictyocaulus, Capillaria, Heterakis, Toxocara, Ascaridia, Oxyuris, Ancylostoma, Uncinaria, Toxascads and Parascaris. Among the trematodes special mention should be made of the family of the Fasciolideae, especially Fasciola hepatica. The special advantage of the compounds of formula I is their efficacy against such parasites that are resistant to benzimidazole-based active ingredients.

Certain Nematodirus, Cooperia and Oesophagostonum species attack the intestinal tract of the host animal whereas others of the species Haemonchus and Ostertagia parasitise in the stomach and those of the species Dictyocaulus parasitise in pulmonary tissue. Parasites of the families Filariidae and Setariidae are found in the internal cell tissue and the organs, for example the heart, the blood vessels, the lymph vessels and the subcutaneous tissue. In this context special mention should be made of dog heartworm, Dirofilaria immitis. The compounds of formula I are highly effective against those parasites.

Furthermore, the compounds of formula I are suitable for controlling parasites that are pathogens of humans, among which, as typical representatives occurring in the digestive tract, mention should be made of those of the species Ancylostoma, Necator, Ascaris, Strongyloides, Trichinella, Capilladia, Trichudis and Enterobius. The compounds of the present invention are also effective against parasites of the species Wuchereria, Brugia, Onchocerca and Loa from the family of the Filariidae, which occur in the blood, in tissue and various organs, and also against Dracunculus and parasites of the species Strongyloides and Trichinella, which infect especially the gastrointestinal tract.

In the area of crop protection, the compounds of formula I are used in unmodified form or, preferably, together with the adjuvants conventionally employed in formulation technology and can therefore be formulated in known manner e.g. into emulsifiable concentrates, directly dilutable solutions, dilute emulsions, soluble powders, granules, and also encapsulations in polymer substances. As with the nature of the compositions, the methods of application are selected in accordance with the intended objectives and the prevailing circumstances.

The invention relates also to pesticides, such as emulsifiable concentrates, suspension concentrates, directly sprayable or dilutable solutions, coatable pastes, dilute emulsions, wettable powders, soluble powders, dispersible powders, dusts, granules or encapsulations in polymer substances, comprising at least one of the active ingredients of the invention, the type of formulation being chosen in accordance with the intended objectives and prevailing circumstances. They are prepared in known manner, e.g. by homogeneously mixing and/or grinding the active ingredients with extenders, for example with solvents, solid carriers, and optionally surface-active compounds (surfactants).

The active ingredient is used in those compositions in pure form: a solid active ingredient, for example, in a specific particle size, or preferably together with at least one of the adjuvants customary in formulation technology, such as extenders, for example solvents or solid carriers, or surface-active compounds (surfactants).

As formulation adjuvants there are used, for example, solid carriers, solvents, stabilisers, “slow release” adjuvants, dyes and optionally surface-active substances (surfactants). Suitable carriers and adjuvants include all those substances customarily used in crop protection products, especially in snail and slug control products. Suitable adjuvants, such as solvents, solid carriers, surface-active compounds, non-ionic surfactants, cationic surfactants, anionic surfactants and other adjuvants in the compositions used according to the invention, include e.g. those described in EP-A-736 252, page 7, line 51, to page 8, line 39. They are included by reference in the subject matter of the present invention.

The compositions for use in crop protection generally comprise from 0.1 to 99% by weight, especially from 0.1 to 95% by weight, of active ingredient and from 1 to 99.9% by weight, especially from 5 to 99.9% by weight, of at least one solid or liquid adjuvant, it generally being possible for from 0 to 25% by weight, especially from 0.1 to 20%, of the compositions to consist of surfactants. Whereas commercial products will preferably be formulated as concentrates, the end user will normally employ dilute formulations, which have much lower active ingredient concentrations. Preferred compositions used in crop protection have especially the following compositions (%=% by weight):

| | |
|---|---|
| Emulsifiable concentrates: | |
| active ingredient: | 1 to 95%, preferably 5 to 20% |
| surfactant: | 1 to 30%, preferably 10 to 20% |
| solvent: | 5 to 98%, preferably 70 to 85% |
| Dusts: | |
| active ingredient: | 0.1 to 10%, preferably 0.1 to 1% |
| solid carrier: | 99.9 to 90%, preferably 99.9 to 99% |
| Suspension concentrates: | |
| active ingredient: | 5 to 75%, preferably 10 to 50% |
| water: | 94 to 24%, preferably 88 to 30% |
| surfactant: | 1 to 40%, preferably 2 to 30% |
| Wettable powders: | |
| active ingredient: | 0.5 to 90%, preferably 1 to 80% |
| surfactant: | 0.5 to 20%, preferably 1 to 15% |
| solid carrier: | 5 to 99%, preferably 15 to 98% |
| Granules: | |
| active ingredient: | 0.5 to 30%, preferably 3 to 15% |
| solid carrier: | 99.5 to 70%, preferably 97 to 85% |

The anthelmintic compositions according to the invention for the control of animal parasites in and on warm-blooded organisms generally comprise from 0.1 to 99% by weight, especially from 0.1 to 95% by weight, of compound of formula I, from 99.9 to 1% by weight, especially from 99.8 to 5% by weight, of a solid or liquid auxiliary, which includes from 0 to 25% by weight, especially from 0.1 to 25% by weight, of a surfactant.

Preferred forms of administration for use in warm-blooded organisms for controlling helminths include solutions, emulsions, suspensions (drenches), feed additives, powders, tablets including effervescent tablets, boli, capsules, micro-encapsulations and pour-on formulations, it being necessary to ensure that the formulation adjuvants are physiologically tolerable.

Suitable solvents in the use of formulations for controlling animal parasites are, for example: alcohols, e.g. ethanol, propanol or butanol, and glycols and ethers and esters thereof, e.g. propylene glycol, dipropylene glycol ether, ethylene glycol, ethylene glycol monomethyl or monoethyl ether, ketones, e.g. cyclohexanone, isophorone or diacetone alcohol, strong polar solvents, e.g. N-methyl-2-pyrrolidone, dimethyl sulfoxide or dimethylformamide or water, vegetable oils, e.g. rapeseed oil, castor oil, coconut oil, sesame oil or soybean oil, and also, where appropriate, silicone oils.

Suitable binders for tablets and boli are chemically modified natural polymeric substances soluble in water or in alcohol, for example starch, cellulose or protein derivatives (e.g. methylcellulose, carboxymethylcellulose, ethyl hydroxyethylcellulose, proteins such as zein, gelatin and the like) and synthetic polymers, e.g. polyvinyl alcohol, polyvinylpyrrolidone etc. Tablets also comprise fillers (e.g. starch, microcrystalline cellulose, sugar, lactose etc.), lubricants and disintegrants.

When the anthelmintic compositions are in the form of feed concentrates, there are used as carriers, for example, performance feeds, feed grains or protein concentrates. Such feed concentrates or compositions can also comprise, besides the active ingredients, auxiliaries, vitamins, antibiotics, chemotherapeutic agents or other pesticides, especially bacteriostatics, fungistatics, coccidiostatics and also hormone preparations, substances having an anabolic action or substances that promote growth, that influence the quality of meat of slaughtered animals or that are useful in some other way for the organism. When the compositions or the compounds of formula I contained therein are added directly to the feed or drinking troughs, the final feed or drinking troughs contain the active ingredients in a concentration of preferably from 0.0005 to 0.02% by weight (5-200 ppm).

The compounds of formula I according to the invention can be used alone or in combination with other biocides. For example, in order to enhance the effect they can be combined with pesticides having the same direction of action or, in order to broaden the spectrum of activity, they can be combined with substances having a different direction of action. It can also be of advantage to add so-called ‘repellents’. Where it is desired to extend the spectrum of activity to endoparasites, e.g. worms, the compounds of formula I are advantageously combined with substances having endoparasiticidal properties. They can, of course, also be used in combination with anti-bacterial agents. Since the compounds of formula I are “adulticides”, i.e. since they are effective especially against the mature stages of target parasites, the addition of pesticides which are more effective against the juvenile stages of the parasites may be very advantageous, since in this way the major portion of those parasites that cause large-scale economic damage will be reached, significantly contributing, moreover, to the avoidance of the formation of resistance. Some combinations may also result in synergistic effects, that is to say the overall amount of active substance used can be reduced, which is desirable from an ecological standpoint. Preferred groups of combination partners and especially preferred combination partners are mentioned hereinbelow; the combinations may comprise, in addition to a compound of formula I, one or more of such partners.

Suitable mixing partners for use in crop protection and for use in controlling endo- and ecto-parasites on warm-blooded organisms are biocides, for example the insecticides and acaricides mentioned hereinbelow and sufficiently known to the person skilled in the art which have a different mechanism of action, for example chitin synthesis inhibitors, growth regulators; active ingredients that act in the same manner as juvenile hormones; active ingredients that act as adulticides; broad-spectrum insecticides, broad-spectrum acaricides, and nematicides; and also the sufficiently known anthelmintics, and substances repelling insects and/or Acarina, the said repellents and detachers.

Examples of suitable insecticides and acaricides are azamethiphos; chlorfenvinphos; cypermethrin, cypermethrin high-cis; cyromazine; diafenthiuron; diazinon; dichlorvos; dicrotophos; dicyclanil; fenoxycarb; fluazuron; furathiocarb; isazofos; jodfenphos; kinoprene; lufenuron; methacriphos; methidathion; monocrotophos; phosphamidon; profenofos; diofenolan; a sub-stance obtainable from the Bacillus thuringiensis strain GC91 or from the strain NCTC11821; pymetrozine; bromopropylate; methoprene; disulfuton; quinalphos; tau-fluvalinate; thiocyclam; thiometon; aldicarb; azinphos-methyl; benfuracarb; bifenthrin; buprofezin; carbofuran; dibutylaminothio; cartap; chlorfluazuron; chlorpyrifos; cyfluthrin; lambda-cyhalothrin; alpha-cypermethrin; zeta-cypermethrin; deltamethrin; diflubenzuron; endosulfan; ethiofencarb; fenitrothion; fenobucarb; fenvalerate; formothion; methiocarb; heptenophos; imidacloprid; isoprocarb; methamidophos; methomyl; mevinphos; parathion; parathion-methyl; phosalone; pirimicarb; propoxur; teflubenzuron; terbufos; triazamate; fenobucarb; tebufenozide; fipronil; beta-cyfluthrin; silafluofen; fenpyroximate; pyridaben; fenazaquin; pyriproxyfen; pyrimidifen; nitenpyram; acetamiprid; avermectin B. (abamectin); emamectin; emamectin benzoate; selamectin, spinosad; a plant extract that is active against insects; a preparation comprising nematodes which is active against insects; a preparation obtainable from Bacillus subtilis; a preparation comprising fungi which is active against insects; a preparation comprising viruses which is active against insects; chlorfenapyr; acephate; acrinathrin; alanycarb; alphamethrin; amitraz; Az 60541; azinphos A; azinphos M; azocyclotin; bendiocarb; bensultap; beta-cyfluthrin; BPMC; brofenprox; bromophos A; bufencarb; butocarboxin; butylpyridaben; cadusafos; carbaryl; carbophenothion; chloethocarb; chlorethoxyfos; chlormephos; cis-resmethrin; clocythrin; clofentezine; cyanophos; cycloprothrin; cyhexatin; demeton M; demeton S; demeton-5-methyl; dichlofenthion; dicliphos; diethion; dimethoate; dimethylvinphos; dioxathion; edifenphos; esfenvalerate; ethion; ethofenprox; ethoprophos; etrimphos; fenamiphos; fenbutatin oxide; fenothiocarb; fenpropathrin; fenpyrad; fenthion; fluazinam; flucycloxuron; flucythrinate; flufenoxuron; flufenprox; fonophos; fosthiazate; fubfenprox; HCH; hexaflumuron; hexythiazox; IKI-220; iprobenfos; isofenphos; isoxathion; ivermectin; malathion; mecarbam; mesulfenphos; metaldehyde; metolcarb; milbemectin; moxidectin; naled; NC 184; omethoate; oxamyl; oxydemeton M; oxydeprofos; permethrin; phenthoate; phorate; phosmet; phoxim; pirimiphos M; pirimiphos E; promecarb; propaphos; prothiofos; prothoate; pyrachlophos; pyrada-phenthion; pyresmethrin; pyrethrum; tebufenozide; salithion; sebufos; sulfotep; sulprofos; tebufenpyrad; tebupirimphos; tefluthrin; temephos; terbam; tetrachlorvinphos; thiacloprid; thiafenox; thiamethoxam; thiodicarb; thiofanox; thionazin; thuringiensin; tralomethrin; triarthene; triazophos; triazuron; trichlorfon; triflumuron; trimethacarb; vamidothion; xylylcarb; Y15301/5302; zetamethrin; DPX-MP062-indoxacarb; methoxyfenozide; bifenazate; XMC (3,5-xylyl methylcarbamate); or the fungus pathogen Metarhizium anisopliae. The mentioned mixing partners are very well known to persons skilled in the art. Most are described in various editions of The Pesticide Manual, The British Crop Protection Council, London, while others are described in various editions of The Merck Index, Merck & Co., Inc., Rahway, N.J., USA, or in the patent literature.

Examples of suitable anthelmintics that can be added to the compositions are mentioned hereinbelow, a number of the examples thereof having, in addition to anthelmintic activity, also an insecticidal and acaricidal activity, some of them already being mentioned in the list above:
(A1) praziguantel=2-cyclohexylcarbonyl-4-oxo-1,2,3,6,7,11 b-hexahydro-4H-pyrazino[2,1-α]isoquinoline
(A2) closantel=3,5-diiodo-N-[5-chloro-2-methyl-4-(a-cyano-4-chlorobenzyl)phenyl]salicyl-amide
(A3) triclabendazole=5-chloro-6-(2,3-dichlorophenoxy)-2-methylthio-1H-benzimidazole
(A4) levamisol=L-(−)-2,3,5,6-tetrahydro-6-phenylimidazo[2,1 b]thiazole
(A5) mebendazole=(5-benzoyl-1H-benzimidazol-2-yl)carbamic acid methyl ester
(A6) omphalotin=a macrocyclic fermentation product of the fungus Omphalotus olearius described in WO 97/20857
(A7) abamectin=avermectin B1
(A8) ivermectin=22,23-dihydroavermectin B1
(A9) moxidectin=5-O-demethyl-28-deoxy-25-(1,3-dimethyl-1-butenyl)-6,28-epoxy-23-(methoxyimino)-milbemycin B
(A10) doramectin=25-cyclohexyl-5-O-demethyl-25-de(1-methylpropyl)-avermectin Ala
(A11) milbemectin=mixture of milbemycin A3 and milbemycin A4
(A12) milbemycinoxim=5-oxime of milbemectin

Examples of suitable repelling substances (repellents and detachers) are for instance
(R1) DEET (N,N-diethyl-m-toluamide)
(R2) KBR 3023 N-butyl-2-oxycarbonyl-(2-hydroxy)-piperidine
(R3) cymiazol=N-2,3-dihydro-3-methyl-1,3-thiazol-2-ylidene-2,4-xylidine

Given what has been said above, a further substantial aspect of the present invention relates to combination preparations for the control of parasites on warm-blooded organisms, which combination preparations comprise, in addition to a compound of formula I, at least one further active ingredient having the same direction of action or a different direction of action and at least one physiologically tolerable carrier. The present invention is not limited to two-component combinations.

The anthelmintic compositions according to the invention generally comprise 0.1 to 99% by weight, in particular 0.1 to 95% by weight, of active ingredient of the formula I or mixtures thereof, and 99.9 to 1% by weight, in particular 99.8 to 5% by weight, of a solid or liquid additive, including 0 to 25% by weight, in particular 0.1 to 25% by weight, of a surfactant.

The compositions according to the invention can be administered to the animals being treated by topical, peroral, parenteral, trans-mucosal or subcutaneous means, the compositions being in the form of solutions, emulsions, suspensions (drenches), powders, tablets, boli, capsules, spray and pour-on formulations.

The pour-on or spot-on method comprises applying the compound of formula I to a locally defined area of the skin or coat, advantageously on the back of the neck or the backbone of the animal. This is carried out, for example, by applying a swab or spray of the pour-on or spot-on formulation to a relatively small area of the coat from where the active ingredient becomes distributed over a wide area of the coat almost automatically as a result of the spreading constituents of the formulation assisted by the movements of the animal.

Pour-on and spot-on formulations advantageously comprise carriers that promote rapid distribution over the surface of the skin or in the coat of the host animal and are generally termed spreading oils. There are suitable, for example, oily solutions; alcoholic and iso-propanolic solutions, e.g. solutions of 2-octyl-dodecanol or oleyl alcohol; solutions in esters of monocarboxylic acids, such as isopropyl myristate, isopropyl palmitate, lauric acid oxalic ester, oleic acid oleyl ester, oleic acid decyl ester, hexyl laurate, oleyl oleate, decyl oleate, capric acid esters of saturated fatty alcohols of chain length C₁₂-C₁₈; solutions of esters of dicarboxylic acids, such as dibutyl phthalate, diisopropyl isophthalate, adipic acid diisopropyl ester, di-n-butyl adipate or solutions of esters of aliphatic acids, e.g. glycols. It may be advantageous for a dispersant known from the pharmaceutical or cosmetic industry also to be present. Examples are 2-pyrrolidone, 2-(N-alkyl)pyrrolidone, acetone, polyethylene glycol and its ethers and esters, propylene glycol or synthetic triglycerides.

Preferred formulations for use in the control of parasites on warm-blooded organisms have the following compositions: (%=% by weight)

### 1. Granules

| | a) | b) |
|---|---|---|
| active ingredient from table 1 | 5% | 10% |
| kaolin | 94% | — |
| highly disperse silicic acid | 1% | — |
| attapulgite | — | 90% |

The active ingredient is dissolved in methylene chloride and sprayed onto the carrier, and the solvent is then evaporated off in vacuo. Such granules can be mixed into the animal feed.

### 2. Granules

| | |
|---|---|
| active ingredient from table 1 | 3% |
| polyethylene glycol (MW 200) | 3% |
| kaolin | 94% |

(MW = molecular weight)

The finely ground active ingredient is uniformly applied, in a mixer, to the kaolin moistened with polyethylene glycol. Non-dusty coated granules are obtained in this manner.

### 3. Tablets and Boli

| | | |
|---|---|---|
| I | active ingredient from table 1 | 33.00% |
| | methylcellulose | 0.80% |
| | highly disperse silicic acid | 0.80% |
| | maize starch | 8.40% |
| II | crystalline lactose | 22.50% |
| | maize starch | 17.00% |
| | microcrystalline cellulose | 16.50% |
| | magnesium stearate | 1.00% |

I Methylcellulose is stirred into water. After the material has swelled, silicic acid is stirred in and the mixture is homogeneously suspended. The active ingredient and maize starch are mixed. The aqueous suspension is incorporated into the resulting mixture and kneaded to a dough. The mass thereby obtained is granulated through a 12 M sieve and dried.
II All 4 excipients are mixed well.
III The premixes obtained according to 1 and 11 are mixed and compressed into tablets or boli.

### 4. Injectable Formulations

A. Oily vehicle (slow release)

| | | |
|---|---|---|
| 1. | active ingredient from table 1 | 0.1-1.0 g |
| | groundnut oil | ad 100 ml |
| 2. | active ingredient from table 1 | 0.1-1.0 g |
| | sesame oil | ad 100 ml |

Preparation: The active ingredient is dissolved in a portion of the oil with stirring and optionally with gentle heating, and after cooling the solution is made up to the desired volume and sterile-filtered through a suitable 0.22 micron membrane filter.

B. Water-miscible solvent (medium rate of release)

| | | |
|---|---|---|
| active ingredient from table 1 | 0.1-1.0 | g |
| 4-hydroxymethyl-1,3-dioxolane (glycerol formal) | 40 | g |
| 1,2-propanediol | ad 100 | ml |
| an active ingredient from table 1 | 0.1-1.0 | g |
| glycerol dimethyl ketal | 40 | g |
| 1,2-propanediol | ad 100 | ml |

Preparation: The active ingredient is dissolved in a portion of the solvent with stirring, and the solution is made up to the desired volume and sterile-filtered through a suitable 0.22 micron membrane filter.

C. Aqueous solubilisate (rapid release)

| | | | |
|---|---|---|---|
| 1. | active ingredient from table 1 | 0.1-1.0 | g |
| | polyethoxylated castor oil (40 ethylene oxide units) | 10 | g |
| | 1,2-propanediol | 20 | g |
| | benzyl alcohol | 1 | g |
| | aqua ad inject. | ad 100 | ml |
| 2. | active ingredient from table 1 | 0.1-1.0 | g |
| | polyethoxylated sorbitan monooleate (20 ethylene | 8 | g |
| | oxide units) | | |
| | 4-hydroxymethyl-1,3-dioxolane (glycerol formal) | 20 | g |
| | benzyl alcohol | 1 | g |
| | aqua ad inject. | ad 100 | ml |

Preparation: The active ingredient is dissolved in the solvents and the surfactant, and the solution is made up to the desired volume with water. Sterile-filtration is then carried out through a suitable membrane filter of 0.22 micron pore diameter.

### 5. Pour-On

A.

| | | |
|---|---|---|
| active ingredient from table 1 | 5 | g |
| isopropyl myristate | 10 | g |
| isopropanol | ad 100 | ml |

B.

| | | |
|---|---|---|
| active ingredient from table 1 | 2 | g |
| hexyl laurate | 5 | g |
| medium-chain triglycerides | 15 | g |
| ethanol | ad 100 | ml |

C.

| | | |
|---|---|---|
| active ingredient from table 1 | 2 | g |
| oleyl oleate | 5 | g |
| N-methyl-pyrrolidone | 40 | g |
| isopropanol | ad 100 | ml |

The aqueous systems may preferably be used also for oral and/or intraruminal administration.

The compositions may also comprise further ingredients such as stabilisers, e.g. vegetable oils and epoxidised vegetable oils (epoxidised coconut oil, rapeseed oil or soybean oil), antifoams, e.g. silicone oil, preservatives, viscosity regulators, binders, tackifiers and fertilisers as well as other active ingredients for obtaining special effects.

It is also possible to add to the described compositions further biologically active substances or additives that have neutral behaviour towards the compounds of formula I and have no adverse effect on the host animal to be treated, and also mineral salts or vitamins.

The compositions according to the invention are prepared in known manner, in the absence of adjuvants, for example by grinding, sieving and/or compressing a solid active ingredient or mixture of active ingredients, for example to a specific particle size, or in the presence of at least one adjuvant, for example by intimately mixing and/or grinding the active ingredient or mixture of active ingredients with the adjuvant(s). The invention relates also to those processes for the preparation of the compositions according to the invention and to the use of the compounds of the formula I in the preparation of those compositions.

The invention relates also to the methods of application of the compositions, i.e. the methods of controlling pests of the mentioned type, such as spraying, atomising, dusting, coating, dressing, scattering or pouring, which are selected in accordance with the intended objectives and prevailing circumstances, and to the use of the compositions for controlling pests of the mentioned type. Typical rates of concentration are from 0.1 to 1000 ppm, preferably from 0.1 to 500 ppm, of active ingredient. The rates of application per hectare are generally from 1 to 2000 g of active ingredient per hectare, especially from 10 to 1000 g/ha, preferably from 20 to 600 g/ha.

A preferred method of application in the area of crop protection is application to the foliage of the plants (foliar application), the number of applications and the rate of application depending on the risk of infestation by the pest in question. However, the active ingredient can also penetrate the plants through the roots (systemic action) if the locus of the plants is impregnated with a liquid formulation or if the active ingredient is incorporated in solid form into the locus of the plants, for example into the soil, e.g. in granular form (soil application). In paddy rice crops, such granules may be applied in metered amounts to the flooded rice field.

The compositions according to the invention are also suitable for protecting plant propagation material, including genetically modified propagation material, e.g. seed material, such as fruit, tubers or grains, or plant cuttings, from animal pests. The propagation material can be treated with the formulation before planting: seed, for example, can be dressed before being sown. The compounds according to the invention can also be applied to grains (coating), either by impregnating the grains with a liquid formulation or by coating them with a solid formulation. The formulation can also be applied to the planting site when the propagation material is being planted, for example to the seed furrow during sowing. The invention relates also to those methods of treating plant propagation material and to the plant propagation material thus treated.

The following Examples serve to illustrate the invention. They do not limit the invention. The symbol ‘h’ stands for ‘hour’.

### PREPARATION EXAMPLES

### N-[2-(2-Chloro-phenoxy)-1,1-dicyano-ethyl]-4-trifluoromethoxy-benzamide

a) To a solution of 6 g aminomalononitrile p-toluenesulfonate in 60 ml pyridine, cooled to 0° C., a solution of 5.32 g 4-trifluoromethoxybenzoyl chloride in 60 ml pyridine is added slowly. The reaction mixture is stirred for 1 h at 0° C. and an additional 2 h at room temperature. After evaporation of the solvent to dryness the residue is dissolved in ethyl acetate and washed successively with a 1 M HCl solution, a saturated solution of sodium bicarbonate, water and brine. The organic phase is separated and dried over magnesium sulfate, filtered and concentrated in vacuo to give N-Dicyanomethyl-4-trifluoromethoxy-benzamide as a beige solid.

b) To a suspension of 29 mg NaH in 5.5 ml dry N-methylpyrrolidone under nitrogen 130 mg N-dicyanomethyl-4-trifluoromethoxy-benzamide are added. The reaction mixture is stirred for 10 min at room temperature, then a solution of 94 mg 1-chloro-2-chloromethoxy-benzene (prepared according to J. Org. Chem. 2003, 68, 1401) in 2.5 ml dry N-methylpyrrolidone is added. The resulting solution is stirred for 5 h at room temperature, then quenched with H₂O and extracted with tert.-butyl-methyl-ether. The organic layer is separated and washed with a saturated solution of sodium bicarbonate, water and brine, then dried with magnesium sulfate, filtered and concentrated in vacuo. The crude compound is purified by preparative reversed phase chromatography on a Daisogel C18-ODS AP column with a water/formic acid (10'000:1) to acetonitrile/formic acid (10'000:1) gradient. The title compound with a melting point of 154-155° C. is isolated as a pale yellow solid by removal of the solvent.

The compounds mentioned in table 1 can be prepared analogously to the procedures described above. The values of the melting points are indicated in ° C.

TABLE 1

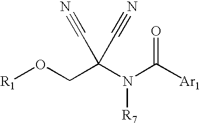

| No | R₁ | R₇ | Ar₁ | mp(° C.) |
|---|---|---|---|---|
| 1.0 | Ph | H | Ph | |
| 1.1 | Ph | H | 4-OCF₃—Ph | |
| 1.2 | 2-Cl—Ph | H | Ph | |
| 1.3 | 2-Cl—Ph | H | 4-OCF₃—Ph | 154-155 |
| 1.4 | 2-Cl—Ph | H | 4-CF₃—Ph | |
| 1.5 | 2-Cl—Ph | H | 4-SCF₃—Ph | |
| 1.6 | 2-Cl—Ph | H | 2-Cl—Ph | |
| 1.7 | 2-Cl—Ph | H | 3-Cl—Ph | |
| 1.8 | 2-Cl—Ph | H | 4-Cl—Ph | |
| 1.9 | 2-Cl—Ph | H | 2,4-Cl₂—Ph | |
| 1.10 | 2-Cl—Ph | H | 3,5-Cl₂—Ph | |
| 1.11 | 2-Cl—Ph | H | 4-Br—Ph | |
| 1.12 | 2-Cl—Ph | H | 4-NO₂—Ph | |
| 1.13 | 2-Cl—Ph | H | 4-CN—Ph | |
| 1.14 | 2-Cl—Ph | H | 4-F—Ph | |
| 1.15 | 2-Cl—Ph | H | 2-Me—Ph | |
| 1.16 | 2-Cl—Ph | H | 3-Me—Ph | |
| 1.17 | 2-Cl—Ph | H | 4-Me—Ph | |
| 1.18 | 2-Cl—Ph | H | 4-OMe—Ph | |
| 1.19 | 2-Cl—Ph | CH₃ | 4-OCF₃—Ph | |
| 1.20 | 2-Cl—Ph | CH₃ | 4-CF₃—Ph | |
| 1.21 | 2-CF₃—Ph | H | Ph | |
| 1.22 | 2-CF₃—Ph | H | 4-OCF₃—Ph | |
| 1.23 | 2-CF₃—Ph | H | 4-CF₃—Ph | |
| 1.24 | 2-CF₃—Ph | H | 4-SCF₃—Ph | |
| 1.25 | 2-CF₃—Ph | H | 2-Cl—Ph | |
| 1.26 | 2-CF₃—Ph | H | 3-Cl—Ph | |
| 1.27 | 2-CF₃—Ph | H | 4-Cl—Ph | |
| 1.28 | 2-CF₃—Ph | H | 2,4-Cl₂—Ph | |
| 1.29 | 2-CF₃—Ph | H | 3,5-Cl₂—Ph | |
| 1.30 | 2-CF₃—Ph | H | 4-Br—Ph | |
| 1.31 | 2-CF₃—Ph | H | 4-NO₂—Ph | |
| 1.32 | 2-CF₃—Ph | H | 4-CN—Ph | |
| 1.33 | 2-CF₃—Ph | H | 4-F—Ph | |
| 1.34 | 2-CF₃—Ph | H | 2-Me—Ph | |
| 1.35 | 2-CF₃—Ph | H | 3-Me—Ph | |
| 1.36 | 2-CF₃—Ph | H | 4-Me—Ph | |
| 1.37 | 2-CF₃—Ph | H | 4-OMe—Ph | |
| 1.38 | 2-CF₃—Ph | CH₃ | 4-OCF₃—Ph | |
| 1.39 | 2-CF₃—Ph | CH₃ | 4-CF₃—Ph | |
| 1.40 | 2-F—Ph | H | Ph | |
| 1.41 | 2-F—Ph | H | 4-OCF₃—Ph | |
| 1.42 | 2-F—Ph | H | 4-CF₃—Ph | |
| 1.43 | 2-F—Ph | H | 4-SCF₃—Ph | |
| 1.44 | 2-F—Ph | H | 2-Cl—Ph | |
| 1.45 | 2-F—Ph | H | 3-Cl—Ph | |
| 1.46 | 2-F—Ph | H | 4-Cl—Ph | |
| 1.47 | 2-F—Ph | H | 2,4-Cl₂—Ph | |
| 1.48 | 2-F—Ph | H | 3,5-Cl₂—Ph | |
| 1.49 | 2-F—Ph | H | 4-Br—Ph | |
| 1.50 | 2-F—Ph | H | 4-NO₂—Ph | |
| 1.51 | 2-F—Ph | H | 4-CN—Ph | |
| 1.52 | 2-F—Ph | H | 4-F—Ph | |
| 1.53 | 2-F—Ph | H | 2-Me—Ph | |
| 1.54 | 2-F—Ph | H | 3-Me—Ph | |
| 1.55 | 2-F—Ph | H | 4-Me—Ph | |
| 1.56 | 2-F—Ph | H | 4-OMe—Ph | |
| 1.57 | 2-F—Ph | CH₃ | 4-OCF₃—Ph | |
| 1.58 | 2-F—Ph | CH₃ | 4-CF₃—Ph | |
| 1.59 | 3-Cl—Ph | H | Ph | |
| 1.60 | 3-Cl—Ph | H | 4-OCF₃—Ph | |
| 1.61 | 3-Cl—Ph | H | 4-CF₃—Ph | |
| 1.62 | 3-Cl—Ph | H | 4-SCF₃—Ph | |
| 1.63 | 3-Cl—Ph | H | 2-Cl—Ph | |
| 1.64 | 3-Cl—Ph | H | 3-Cl—Ph | |
| 1.65 | 3-Cl—Ph | H | 4-Cl—Ph | |
| 1.66 | 3-Cl—Ph | H | 2,4-Cl₂—Ph | |
| 1.67 | 3-Cl—Ph | H | 3,5-Cl₂—Ph | |
| 1.68 | 3-Cl—Ph | H | 4-Br—Ph | |
| 1.69 | 3-Cl—Ph | H | 4-NO₂—Ph | |
| 1.70 | 3-Cl—Ph | H | 4-CN—Ph | |
| 1.71 | 3-Cl—Ph | H | 4-F—Ph | |
| 1.72 | 3-Cl—Ph | H | 2-Me—Ph | |
| 1.73 | 3-Cl—Ph | H | 3-Me—Ph | |
| 1.74 | 3-Cl—Ph | H | 4-Me—Ph | |
| 1.75 | 3-Cl—Ph | H | 4-OMe—Ph | |
| 1.76 | 3-Cl—Ph | CH₃ | 4-OCF₃—Ph | |
| 1.77 | 3-Cl—Ph | CH₃ | 4-CF₃—Ph | |
| 1.78 | 4-Cl—Ph | H | Ph | |
| 1.79 | 4-Cl—Ph | H | 4-OCF₃—Ph | 157-159 |
| 1.80 | 4-Cl—Ph | H | 4-CF₃—Ph | |
| 1.81 | 4-Cl—Ph | H | 4-SCF₃—Ph | |
| 1.82 | 4-Cl—Ph | H | 2-Cl—Ph | |
| 1.83 | 4-Cl—Ph | H | 3-Cl—Ph | |
| 1.84 | 4-Cl—Ph | H | 4-Cl—Ph | |
| 1.85 | 4-Cl—Ph | H | 2,4-Cl₂—Ph | |
| 1.86 | 4-Cl—Ph | H | 3,5-Cl₂—Ph | |
| 1.87 | 4-Cl—Ph | H | 4-Br—Ph | |
| 1.88 | 4-Cl—Ph | H | 4-NO₂—Ph | |
| 1.89 | 4-Cl—Ph | H | 4-CN—Ph | |
| 1.90 | 4-Cl—Ph | H | 4-F—Ph | |
| 1.91 | 4-Cl—Ph | H | 2-Me—Ph | |
| 1.92 | 4-Cl—Ph | H | 3-Me—Ph | |
| 1.93 | 4-Cl—Ph | H | 4-Me—Ph | |
| 1.94 | 4-Cl—Ph | H | 4-OMe—Ph | |
| 1.95 | 4-Cl—Ph | CH₃ | 4-OCF₃—Ph | |
| 1.96 | 4-Cl—Ph | CH₃ | 4-CF₃—Ph | |
| 1.97 | 2,4-Cl₂—Ph | H | Ph | |
| 1.98 | 2,4-Cl₂—Ph | H | 4-OCF₃—Ph | 158-160 |
| 1.99 | 2,4-Cl₂—Ph | H | 4-CF₃—Ph | |
| 1.100 | 2,4-Cl₂—Ph | H | 4-SCF₃—Ph | |
| 1.101 | 2,4-Cl₂—Ph | H | 2-Cl—Ph | |
| 1.102 | 2,4-Cl₂—Ph | H | 3-Cl—Ph | |
| 1.103 | 2,4-Cl₂—Ph | H | 4-Cl—Ph | |
| 1.104 | 2,4-Cl₂—Ph | H | 2,4-Cl₂—Ph | |
| 1.105 | 2,4-Cl₂—Ph | H | 3,5-Cl₂—Ph | |
| 1.106 | 2,4-Cl₂—Ph | H | 4-Br—Ph | |
| 1.107 | 2,4-Cl₂—Ph | H | 4-NO2—Ph | |
| 1.108 | 2,4-Cl₂—Ph | H | 4-CN—Ph | |
| 1.109 | 2,4-Cl₂—Ph | H | 4-F—Ph | |
| 1.110 | 2,4-Cl₂—Ph | H | 2-Me—Ph | |
| 1.111 | 2,4-Cl₂—Ph | H | 3-Me—Ph | |
| 1.112 | 2,4-Cl₂—Ph | H | 4-Me—Ph | |
| 1.113 | 2,4-Cl₂—Ph | H | 4-OMe—Ph | |
| 1.114 | 2,4-Cl₂—Ph | CH₃ | 4-OCF₃—Ph | |
| 1.115 | 2,4-Cl₂—Ph | CH₃ | 4-CF₃—Ph | |
| 1.116 | 2-OMe-4-CN—Ph | H | Ph | |
| 1.117 | 2-OMe-4-CN—Ph | H | 4-OCF₃—Ph | |
| 1.118 | 2-OMe-4-CN—Ph | H | 4-CF₃—Ph | |
| 1.119 | 2-OMe-4-CN—Ph | H | 4-SCF₃—Ph | |
| 1.120 | 2-OMe-4-CN—Ph | H | 2-Cl—Ph | |
| 1.121 | 2-OMe-4-CN—Ph | H | 3-Cl—Ph | |
| 1.122 | 2-OMe-4-CN—Ph | H | 4-Cl—Ph | |
| 1.123 | 2-OMe-4-CN—Ph | H | 2,4-Cl₂—Ph | |
| 1.124 | 2-OMe-4-CN—Ph | H | 3,5-Cl₂—Ph | |
| 1.125 | 2-OMe-4-CN—Ph | H | 4-Br—Ph | |
| 1.126 | 2-OMe-4-CN—Ph | H | 4-NO₂—Ph | |
| 1.127 | 2-OMe-4-CN—Ph | H | 4-CN—Ph | |
| 1.128 | 2-OMe-4-CN—Ph | H | 4-F—Ph | |
| 1.129 | 2-OMe-4-CN—Ph | H | 2-Me—Ph | |
| 1.130 | 2-OMe-4-CN—Ph | H | 3-Me—Ph | |
| 1.131 | 2-OMe-4-CN—Ph | H | 4-Me—Ph | |
| 1.132 | 2-OMe-4-CN—Ph | H | 4-OMe—Ph | |
| 1.133 | 2-OMe-4-CN—Ph | CH₃ | 4-OCF₃—Ph | |
| 1.134 | 2-OMe-4-CN—Ph | CH₃ | 4-CF₃—Ph | |
| 1.135 | 2-OMe—Ph | H | Ph | |
| 1.136 | 2-OMe—Ph | H | 4-OCF₃—Ph | 69-71 |
| 1.137 | 2-OMe—Ph | H | 4-CF₃—Ph | |
| 1.138 | 2-OMe—Ph | H | 4-SCF₃—Ph | |
| 1.139 | 2-OMe—Ph | H | 2-Cl—Ph | |
| 1.140 | 2-OMe—Ph | H | 3-Cl—Ph | |
| 1.141 | 2-OMe—Ph | H | 4-Cl—Ph | |
| 1.142 | 2-OMe—Ph | H | 2,4-Cl₂—Ph | |
| 1.143 | 2-OMe—Ph | H | 3,5-Cl₂—Ph | |
| 1.144 | 2-OMe—Ph | H | 4-Br—Ph | |
| 1.145 | 2-OMe—Ph | H | 4-NO₂—Ph | |
| 1.146 | 2-OMe—Ph | H | 4-CN—Ph | |
| 1.147 | 2-OMe—Ph | H | 4-F—Ph | |
| 1.148 | 2-OMe—Ph | H | 2-Me—Ph | |
| 1.149 | 2-OMe—Ph | H | 3-Me—Ph | |
| 1.150 | 2-OMe—Ph | H | 4-Me—Ph | |
| 1.151 | 2-OMe—Ph | H | 4-OMe—Ph | |
| 1.152 | 2-OMe—Ph | CH₃ | 4-OCF₃—Ph | |
| 1.153 | 2-OMe—Ph | CH₃ | 4-CF₃—Ph | |
| 1.154 | 3-OMe—Ph | H | Ph | |
| 1.155 | 3-OMe—Ph | H | 4-OCF₃—Ph | |
| 1.156 | 3-OMe—Ph | H | 4-CF₃—Ph | |
| 1.157 | 3-OMe—Ph | H | 4-SCF₃—Ph | |
| 1.158 | 3-OMe—Ph | H | 2-Cl—Ph | |
| 1.159 | 3-OMe—Ph | H | 3-Cl—Ph | |
| 1.160 | 3-OMe—Ph | H | 4-Cl—Ph | |
| 1.161 | 3-OMe—Ph | H | 2,4-Cl₂—Ph | |
| 1.162 | 3-OMe—Ph | H | 3,5-Cl₂—Ph | |
| 1.163 | 3-OMe—Ph | H | 4-Br—Ph | |
| 1.164 | 3-OMe—Ph | H | 4-NO₂—Ph | |
| 1.165 | 3-OMe—Ph | H | 4-CN—Ph | |
| 1.166 | 3-OMe—Ph | H | 4-F—Ph | |
| 1.167 | 3-OMe—Ph | H | 2-Me—Ph | |
| 1.168 | 3-OMe—Ph | H | 3-Me—Ph | |
| 1.169 | 3-OMe—Ph | H | 4-Me—Ph | |
| 1.170 | 3-OMe—Ph | H | 4-OMe—Ph | |
| 1.171 | 3-OMe—Ph | CH₃ | 4-OCF₃—Ph | |
| 1.172 | 3-OMe—Ph | CH₃ | 4-CF₃—Ph | |
| 1.173 | 4-OMe—Ph | H | Ph | |
| 1.174 | 4-OMe—Ph | H | 4-OCF₃—Ph | |
| 1.175 | 4-OMe—Ph | H | 4-CF₃—Ph | |
| 1.176 | 4-OMe—Ph | H | 4-SCF₃—Ph | |
| 1.177 | 4-OMe—Ph | H | 2-Cl—Ph | |
| 1.178 | 4-OMe—Ph | H | 3-Cl—Ph | |
| 1.179 | 4-OMe—Ph | H | 4-Cl—Ph | |
| 1.180 | 4-OMe—Ph | H | 2,4-Cl₂—Ph | |
| 1.181 | 4-OMe—Ph | H | 3,5-Cl₂—Ph | |
| 1.182 | 4-OMe—Ph | H | 4-Br—Ph | |
| 1.183 | 4-OMe—Ph | H | 4-NO₂—Ph | |
| 1.184 | 4-OMe—Ph | H | 4-CN—Ph | |
| 1.185 | 4-OMe—Ph | H | 4-F—Ph | |
| 1.186 | 4-OMe—Ph | H | 2-Me—Ph | |
| 1.187 | 4-OMe—Ph | H | 3-Me—Ph | |
| 1.188 | 4-OMe—Ph | H | 4-Me—Ph | |
| 1.189 | 4-OMe—Ph | H | 4-OMe—Ph | |
| 1.190 | 4-OMe—Ph | CH₃ | 4-OCF₃—Ph | |
| 1.191 | 4-OMe—Ph | CH₃ | 4-CF₃—Ph | |
| 1.192 | 2-CF₃-4,5-F₂—Ph | H | Ph | |
| 1.193 | 2-CF₃-4,5-F₂—Ph | H | 4-OCF₃—Ph | |
| 1.194 | 2-CF₃-4,5-F₂—Ph | H | 4-CF₃—Ph | |
| 1.195 | 2-CF₃-4,5-F₂—Ph | H | 4-SCF₃—Ph | |
| 1.196 | 2-CF₃-4,5-F₂—Ph | H | 2-Cl—Ph | |
| 1.197 | 2-CF₃-4,5-F₂—Ph | H | 3-Cl—Ph | |
| 1.198 | 2-CF₃-4,5-F₂—Ph | H | 4-Cl—Ph | |
| 1.199 | 2-CF₃-4,5-F₂—Ph | H | 2,4-Cl₂—Ph | |
| 1.200 | 2-CF₃-4,5-F₂—Ph | H | 3,5-Cl₂—Ph | |
| 1.201 | 2-CF₃-4,5-F₂—Ph | H | 4-Br—Ph | |
| 1.202 | 2-CF₃-4,5-F₂—Ph | H | 4-NO₂—Ph | |
| 1.203 | 2-CF₃-4,5-F₂—Ph | H | 4-CN—Ph | |
| 1.204 | 2-CF₃-4,5-F₂—Ph | H | 4-F—Ph | |
| 1.205 | 2-CF₃-4,5-F₂—Ph | H | 2-Me—Ph | |
| 1.206 | 2-CF₃-4,5-F₂—Ph | H | 3-Me—Ph | |
| 1.207 | 2-CF₃-4,5-F₂—Ph | H | 4-Me—Ph | |
| 1.208 | 2-CF₃-4,5-F₂—Ph | H | 4-OMe—Ph | |
| 1.209 | 2-CF₃-4,5-F₂—Ph | CH₃ | 4-OCF₃—Ph | |
| 1.210 | 2-CF₃-4,5-F₂—Ph | CH₃ | 4-CF₃—Ph | |
| 1.211 | 2-CF₃-4-CN—Ph | H | Ph | |
| 1.212 | 2-CF₃-4-CN—Ph | H | 4-OCF₃—Ph | |
| 1.213 | 2-CF₃-4-CN—Ph | H | 4-CF₃—Ph | |
| 1.214 | 2-CF₃-4-CN—Ph | H | 4-SCF₃—Ph | |
| 1.215 | 2-CF₃-4-CN—Ph | H | 2-Cl—Ph | |
| 1.216 | 2-CF₃-4-CN—Ph | H | 3-Cl—Ph | |
| 1.217 | 2-CF₃-4-CN—Ph | H | 4-Cl—Ph | |
| 1.218 | 2-CF₃-4-CN—Ph | H | 2,4-Cl₂—Ph | |
| 1.219 | 2-CF₃-4-CN—Ph | H | 3,5-Cl₂—Ph | |
| 1.220 | 2-CF₃-4-CN—Ph | H | 4-Br—Ph | |
| 1.221 | 2-CF₃-4-CN—Ph | H | 4-NO₂—Ph | |
| 1.222 | 2-CF₃-4-CN—Ph | H | 4-CN—Ph | |
| 1.223 | 2-CF₃-4-CN—Ph | H | 4-F—Ph | |
| 1.224 | 2-CF₃-4-CN—Ph | H | 2-Me—Ph | |
| 1.225 | 2-CF₃-4-CN—Ph | H | 3-Me—Ph | |
| 1.226 | 2-CF₃-4-CN—Ph | H | 4-Me—Ph | |
| 1.227 | 2-CF₃-4-CN—Ph | H | 4-OMe—Ph | |
| 1.228 | 2-CF₃-4-CN—Ph | CH₃ | 4-OCF₃—Ph | |
| 1.229 | 2-CF₃-4-CN—Ph | CH₃ | 4-CF₃—Ph | |
| 1.230 | 2-CF₃-5-CN—Ph | H | Ph | |
| 1.231 | 2-CF₃-5-CN—Ph | H | 4-OCF₃—Ph | |
| 1.232 | 2-CF₃-5-CN—Ph | H | 4-CF₃—Ph | |
| 1.233 | 2-CF₃-5-CN—Ph | H | 4-SCF₃—Ph | |
| 1.234 | 2-CF₃-5-CN—Ph | H | 2-Cl—Ph | |
| 1.235 | 2-CF₃-5-CN—Ph | H | 3-Cl—Ph | |
| 1.236 | 2-CF₃-5-CN—Ph | H | 4-Cl—Ph | |
| 1.237 | 2-CF₃-5-CN—Ph | H | 2,4-Cl₂—Ph | |
| 1.238 | 2-CF₃-5-CN—Ph | H | 3,5-Cl₂—Ph | |
| 1.239 | 2-CF₃-5-CN—Ph | H | 4-Br—Ph | |
| 1.240 | 2-CF₃-5-CN—Ph | H | 4-NO₂—Ph | |
| 1.241 | 2-CF₃-5-CN—Ph | H | 4-CN—Ph | |
| 1.242 | 2-CF₃-5-CN—Ph | H | 4-F—Ph | |
| 1.243 | 2-CF₃-5-CN—Ph | H | 2-Me—Ph | |
| 1.244 | 2-CF₃-5-CN—Ph | H | 3-Me—Ph | |
| 1.245 | 2-CF₃-5-CN—Ph | H | 4-Me—Ph | |
| 1.246 | 2-CF₃-5-CN—Ph | H | 4-OMe—Ph | |
| 1.247 | 2-CF₃-5-CN—Ph | CH₃ | 4-OCF₃—Ph | |
| 1.248 | 2-CF₃-5-CN—Ph | CH₃ | 4-CF₃—Ph | |
| 1.249 | 2-(2,4-Cl₂—PhO)-5-Cl—Ph | H | Ph | |
| 1.250 | 2-(2,4-Cl₂—PhO)-5-Cl—Ph | H | 4-OCF₃—Ph | |
| 1.251 | 2-(2,4-Cl₂—PhO)-5-Cl—Ph | H | 4-CF₃—Ph | |
| 1.252 | 2-(2,4-Cl₂—PhO)-5-Cl—Ph | H | 4-SCF₃—Ph | |
| 1.253 | 2-(2,4-Cl₂—PhO)-5-Cl—Ph | H | 2-Cl—Ph | |
| 1.254 | 2-(2,4-Cl₂—PhO)-5-Cl—Ph | H | 3-Cl—Ph | |
| 1.255 | 2-(2,4-Cl₂—PhO)-5-Cl—Ph | H | 4-Cl—Ph | |
| 1.256 | 2-(2,4-Cl₂—PhO)-5-Cl—Ph | H | 2,4-Cl₂—Ph | |
| 1.257 | 2-(2,4-Cl₂—PhO)-5-Cl—Ph | H | 3,5-Cl₂—Ph | |
| 1.258 | 2-(2,4-Cl₂—PhO)-5-Cl—Ph | H | 4-Br—Ph | |
| 1.259 | 2-(2,4-Cl₂—PhO)-5-Cl—Ph | H | 4-NO₂—Ph | |
| 1.260 | 2-(2,4-Cl₂—PhO)-5-Cl—Ph | H | 4-CN—Ph | |
| 1.261 | 2-(2,4-Cl₂—PhO)-5-Cl—Ph | H | 4-F—Ph | |
| 1.262 | 2-(2,4-Cl₂—PhO)-5-Cl—Ph | H | 2-Me—Ph | |
| 1.263 | 2-(2,4-Cl₂—PhO)-5-Cl—Ph | H | 3-Me—Ph | |
| 1.264 | 2-(2,4-Cl₂—PhO)-5-Cl—Ph | H | 4-Me—Ph | |
| 1.265 | 2-(2,4-Cl₂—PhO)-5-Cl—Ph | H | 4-OMe—Ph | |
| 1.266 | 2-(2,4-Cl₂—PhO)-5-Cl—Ph | CH₃ | 4-OCF₃—Ph | |
| 1.267 | 2-(2,4-Cl₂—PhO)-5-Cl—Ph | CH₃ | 4-CF₃—Ph | |
| 1.268 | 2-(2,4-Cl₂—PhO)-5-CN—Ph | H | Ph | |
| 1.269 | 2-(2,4-Cl₂—PhO)-5-CN—Ph | H | 4-OCF₃—Ph | 166-167 |
| 1.270 | 2-(2,4-Cl₂—PhO)-5-CN—Ph | H | 4-CF₃—Ph | |
| 1.271 | 2-(2,4-Cl₂—PhO)-5-CN—Ph | H | 4-SCF₃—Ph | |
| 1.272 | 2-(2,4-Cl₂—PhO)-5-CN—Ph | H | 2-Cl—Ph | |
| 1.273 | 2-(2,4-Cl₂—PhO)-5-CN—Ph | H | 3-Cl—Ph | |
| 1.274 | 2-(2,4-Cl₂—PhO)-5-CN—Ph | H | 4-Cl—Ph | |
| 1.275 | 2-(2,4-Cl₂—PhO)-5-CN—Ph | H | 2,4-Cl₂—Ph | |
| 1.276 | 2-(2,4-Cl₂—PhO)-5-CN—Ph | H | 3,5-Cl₂—Ph | |
| 1.277 | 2-(2,4-Cl₂—PhO)-5-CN—Ph | H | 4-Br—Ph | |
| 1.278 | 2-(2,4-Cl₂—PhO)-5-CN—Ph | H | 4-NO₂—Ph | |
| 1.279 | 2-(2,4-Cl₂—PhO)-5-CN—Ph | H | 4-CN—Ph | |
| 1.280 | 2-(2,4-Cl₂—PhO)-5-CN—Ph | H | 4-F—Ph | |
| 1.281 | 2-(2,4-Cl₂—PhO)-5-CN—Ph | H | 2-Me—Ph | |
| 1.282 | 2-(2,4-Cl₂—PhO)-5-CN—Ph | H | 3-Me—Ph | |
| 1.283 | 2-(2,4-Cl₂—PhO)-5-CN—Ph | H | 4-Me—Ph | |
| 1.284 | 2-(2,4-Cl₂—PhO)-5-CN—Ph | H | 4-OMe—Ph | |
| 1.285 | 2-(2,4-Cl₂—PhO)-5-CN—Ph | CH₃ | 4-OCF₃—Ph | |
| 1.286 | 2-(2,4-Cl₂—PhO)-5-CN—Ph | CH₃ | 4-CF₃—Ph | |
| 1.287 | 2-(2,4-Cl₂—PhO)-4-CN—Ph | H | Ph | |
| 1.288 | 2-(2,4-Cl₂—PhO)-4-CN—Ph | H | 4-OCF₃—Ph | |
| 1.289 | 2-(2,4-Cl₂—PhO)-4-CN—Ph | H | 4-CF₃—Ph | |
| 1.290 | 2-(2,4-Cl₂—PhO)-4-CN—Ph | H | 4-SCF₃—Ph | |
| 1.291 | 2-(2,4-Cl₂—PhO)-4-CN—Ph | H | 2-Cl—Ph | |
| 1.292 | 2-(2,4-Cl₂—PhO)-4-CN—Ph | H | 3-Cl—Ph | |
| 1.293 | 2-(2,4-Cl₂—PhO)-4-CN—Ph | H | 4-Cl—Ph | |
| 1.294 | 2-(2,4-Cl₂—PhO)-4-CN—Ph | H | 2,4-Cl₂—Ph | |
| 1.295 | 2-(2,4-Cl₂—PhO)-4-CN—Ph | H | 3,5-Cl₂—Ph | |
| 1.296 | 2-(2,4-Cl₂—PhO)-4-CN—Ph | H | 4-Br—Ph | |
| 1.297 | 2-(2,4-Cl₂—PhO)-4-CN—Ph | H | 4-NO₂—Ph | |
| 1.298 | 2-(2,4-Cl₂—PhO)-4-CN—Ph | H | 4-CN—Ph | |
| 1.299 | 2-(2,4-Cl₂—PhO)-4-CN—Ph | H | 4-F—Ph | |
| 1.300 | 2-(2,4-Cl₂—PhO)-4-CN—Ph | H | 2-Me—Ph | |
| 1.301 | 2-(2,4-Cl₂—PhO)-4-CN—Ph | H | 3-Me—Ph | |
| 1.302 | 2-(2,4-Cl₂—PhO)-4-CN—Ph | H | 4-Me—Ph | |
| 1.303 | 2-(2,4-Cl₂—PhO)-4-CN—Ph | H | 4-OMe—Ph | |
| 1.304 | 2-(2,4-Cl₂—PhO)-4-CN—Ph | CH₃ | 4-OCF₃—Ph | |
| 1.305 | 2-(2,4-Cl₂—PhO)-4-CN—Ph | CH₃ | 4-CF₃—Ph | |

The oily solutions include e.g. vegetable oils, such as olive oil, groundnut oil, sesame oil, pine oil, linseed oil and castor oil. The vegetable oils may also be in epoxidised form. It is also possible to use paraffins and silicone oils.

Generally a pour-on or spot-on formulation will contain from 1 to 20% by weight of a compound of formula I, from 0.1 to 50% by weight dispersant and from 45 to 98.9% by weight solvent.

The pour-on or spot-on method can be used especially advantageously for herd animals, such as cattle, horses, sheep and pigs, where it is difficult or time-consuming to treat all the animals orally or via injection. By virtue of its simplicity, this method can of course also be used for all other animals, including individual domestic animals and pets, and is very popular with the keepers of the animals because it can often be carried out without the expert assistance of a veterinary surgeon.

Whereas commercial products will preferably be formulated as concentrates, the end user will normally employ dilute formulations.

Such formulations may also comprise further ingredients, such as stabilisers, antifoams, viscosity regulators, binders and tackifiers as well as other active ingredients for obtaining special effects.

The present invention relates also to such anthelmintic compositions employed by the end user.

In each of the methods according to the invention for controlling pests and in each of the pesticidal compositions according to the invention, the active ingredients of formula I can be used in any of their spatial configurations or mixtures thereof.

The invention also encompasses a method for the prophylactic protection of warm-blooded organisms, especially of productive livestock, domestic animals and pets, against parasitic helminths, which method comprises administering to the animals the active ingredient of formula I or active ingredient formulations prepared therefrom as an additive to the feed or to the drinking troughs or in solid or liquid form orally, by injection or parenterally. The invention also encompasses the compounds of formula I according to the invention for use in one of the mentioned methods.

The Examples that follow serve merely to illustrate the invention without limiting the invention, the expression ‘active ingredient’ denoting one of the substances listed in the Tables.

### BIOLOGICAL EXAMPLES

### A. Control of Animal Parasites

### Example B.1

### In-Vivo Test against Trichostronqylus colubriformis and Haemonchus contortus in Mongolian gerbils (Merones unquiculatus) on Peroral Administration

Gerbils are artificially infected by gavage with ca. 2000 third instar larvae each of T. colubriformis and H. contortus seven, respectively six days before treatment. Treatment is performed orally with the formulated test compound at doses ranging normally from 0.1 mg/kg to 100 mg/kg. Three days after treatment, gerbils are euthanised and dissected to recover H. contortus from the stomach and T. colubriformis from the upper part of the midgut. Efficacy is expressed as a % reduction in worm numbers in comparison with a placebo treated group, using the Abbot's formula.

A strong reduction of the nematode attack is achieved in this test with compounds of the formula I. For example, compounds 1.3, 1.269 and 1.136 showed more than 80% efficacy at 32 mg/kg p.o. on both species of worms.

### B. Control of Plant Pests

### Example B.2

### Action against Heliothis virescens Caterpillars

Young soybean plants are sprayed with an aqueous emulsion spray mixture comprising 400 ppm of active ingredient and, after the spray-coating has dried, are populated with 10 caterpillars of Heliothis virescens in the first stage and then placed in a plastics container. Evaluation is carried out 6 days later. The percentage reduction in population and in feeding damage (% activity) are determined by comparing the number of dead caterpillars and the feeding damage on the treated plants with that on untreated plants.

The compounds of the Tables exhibit good activity against Heliothis virescens in this test.

### Example B.3

### Action Against Plutella xylostella Caterpillars

Young cabbage plants are sprayed with an aqueous emulsion spray mixture comprising 400 ppm of active ingredient and, after the spray-coating has dried, are populated with 10 caterpillars of Plutella xylostella in the third stage and then placed in a plastics container. Evaluation is carried out 3 days later. The percentage reduction in population and in feeding damage (% activity) are determined by comparing the number of dead caterpillars and the feeding damage on the treated plants with that on untreated plants.

The compounds of the Tables exhibit good activity against Plutella xylostella.

### Example B.4

### Action Against Diabrotica balteata Larvae

Maize seedlings are sprayed with an aqueous emulsion spray mixture comprising 400 ppm of active ingredient and, after the spray-coating has dried, are populated with 10 larvae of Diabrotica balteata in the second stage and then placed in a plastics container. Evaluation is carried out 6 days later. The percentage reduction in population (% activity) is determined by comparing the number of dead larvae on the treated plants with that on untreated plants.

The compounds of the Tables exhibit good activity against Diabrotica balteata in this test.

1-25. (canceled)

26. A compound of the formula 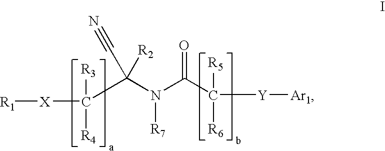
wherein
R₁ is hydrogen, C₁-C₆-alkyl, aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉, COOR₈, unsubstituted or one- to five-fold substituted aryl, unsubstituted or one- to five-fold substituted aryloxy, unsubstituted or one- to five-fold substituted arylthio, unsubstituted or one- to five-fold substituted hetaryl or unsubstituted or one- to five-fold substituted hetaryloxy, the substituents selected from the group consisting of halogen, cyano, NO₂, hydroxy, C₁-C₆-alkyl, halo-C₁-C₆-alkyl, C₁-C₆-alkoxy, halo-C₁-C₆-alkoxy, NH₂, C₁-C₆-alkylamino, di-C₁-C₆-alkylamino, C₁-C₆-alkylthio, COR₈, CONR₈R₉ and COOR₈;
R₂ is cyano, CONR₈R₉ or COOR₈;
R₃, R₄, R₅ and R₆, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
R₇ is hydrogen, C₁-C₆-alkyl, C₂-C₆-alkenyl, C₂-C₆-alkynyl or C₃-C₆-cycloalkyl each optionally substituted with one or more substituents selected from the group consisting of halogen, cyano, hydroxy and C₁-C₆-alkoxy; or C₁-C₆-alkylcarbonyl or C₁-C₆-alkoxycarbonyl;
R₈ and R₉, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
or, R₈ and R₉, together with the nitrogen atom to which they are attached, form a ring of 3 to 6 atoms, optionally including one additional atom of nitrogen, sulfur or oxygen, optionally substituted with 1 to 4 substituents selected form the group consisting of halogen, CN, NO₂, hydroxy, C₁-C₆-alkyl and C₁-C₆-alkoxy;
Ar₁ is aryl or hetaryl each optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉ and COOR₈;
X and Y are, independently of one another, a direct bond or oxygen; and
a and b, independently of one another, are 0, 1 or 2; and
enantiomers and/or salts thereof.

27. The compound of claim 26, wherein R1 is aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₂NR₈R₉, unsubstituted or one- to five-fold substituted aryl, unsubstituted or one- to five-fold substituted aryloxy, or unsubstituted or one- to five-fold substituted arylthio, the substituents selected from the group consisting of halogen, cyano, C₁-C₆-alkyl, halo-C₁-C₆-alkyl, C₁-C₆-alkoxy and halo-C₁-C₆-alkoxy.

28. The compound of claim 26, wherein R₁ is aryl optionally substituted with one or more substitutents selected from the group consisting of halogen, cyano, C₁-C₄-alkyl, C₂-C₄-alkenyl, halo-C₁-C₄-alkyl, halo-C₂-C₄-alkenyl, C₁-C₄-alkoxy, C₂-C₄-alkenyloxy, halo-C₁-C₄-alkoxy, halo-C₂-C₄-alkenyloxy, C₁-C₄-alkylthio, halo-C₁-C₄-alkylthio, unsubstituted or one- to five-fold substituted aryl, unsubstituted or one- to five-fold substituted aryloxy, or unsubstituted or one- to five-fold substituted arylthio, the substituents selected from the group consisting of halogen, cyano, C₁-C₆-alkyl, halo-C₁-C₆-alkyl, C₁-C₆-alkoxy and halo-C₁-C₆-alkoxy.

29. The compound of claim 26, wherein R₁ is phenyl optionally substituted with one or more substitutents selected from the group consisting of halogen, cyano, C₁-C₂-alkyl, halo-C₁-C₂-alkyl, C₁-C₂-alkoxy, halo-C₁-C₂-alkoxy, C₁-C₂-alkylthio, halo-C₁-C₂-alkylthio, unsubstituted or one- to five-fold substituted phenoxy, or unsubstituted or one- to five-fold substituted arylthio, the substituents selected from the group consisting of halogen, cyano, C₁-C₄-alkyl, halo-C₁-C₄-alkyl, C₁-C₄-alkoxy and halo-C₁-C₄-alkoxy.

30. The compound of claim 26, wherein R₁ is phenyl optionally substituted with one or more substitutents selected from the group consisting of halogen, cyano, methyl, halo-methyl, methoxy or unsubstituted or mono- or dihalogen substituted phenoxy.

31. The compound of claim 26, wherein R₂ is cyano.

32. The compound of claim 26, wherein R₃, R₄, R₅ and R₆, independently of one another, are hydrogen or C₁-C₄-alkyl, in particular hydrogen.

33. The compound of claim 26, wherein R₇ is hydrogen or methyl, in particular hydrogen.

34. The compound of claim 26, wherein Ar₁ is aryl optionally substituted with one or more substitutents selected from the group consisting of halogen, cyano, NO₂, C₁-C₆-alkyl, C₂-C₆-alkenyl, halo-C₁-C₆-alkyl, C₁-C₆-alkoxy, C₂-C₆-alkenyloxy, halo-C₁-C₆-alkoxy, halo-C₂-C₆-alkenyloxy, C₁-C₆-alkylthio, halo-C₁-C₆-alkylthio, C₁-C₆-alkylsulfonyl, halo-C₁-C₆-alkylsulfonyl, C₁-C₆-alkylamino and di-C₁-C₆-alkylamino.

35. The compound of claim 26, wherein Ar₁ is phenyl optionally substituted with one or more substitutents selected from the group consisting of halogen, cyano, NO₂, C₁-C₄-alkyl, halo-C₁-C₄-alkyl, C₁-C₄-alkoxy, halo-C₁-C₄-alkoxy, C₁-C₄-alkylthio and halo-C₁-C₄-alkylthio.

36. The compound of claim 26, wherein Ar₁ is phenyl optionally substituted with one or more substitutents selected from the group consisting of halogen, cyano, NO₂, C₁-C₂-alkyl, halo-C₁-C₂-alkyl, C₁-C₂-alkoxy, halo-C₁-C₂-alkoxy, C₁-C₂-alkylthio and halo-C₁-C₂-alkylthio.

37. The compound of claim 26, wherein X is oxygen.

38. The compound of claim 26, wherein Y is a direct bond.

39. The compound of claim 26, wherein a is 1 and b is 0.

40. The compound of claim 26, wherein R1 is aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₂NR₈R₉, unsubstituted or one- to five-fold substituted aryl, unsubstituted or one- to five-fold substituted aryloxy, or unsubstituted or one- to five-fold substituted arylthio, the substituents selected from the group consisting of halogen, cyano, C₁-C₆-alkyl, halo-C₁-C₆-alkyl, C₁-C₆-alkoxy and halo-C₁-C₆-alkoxy;
R₂ is cyano or COOR₈;
R₃, R₄, R₅ and R₆, independently of one another, are hydrogen or C₁-C₆-alkyl, each optionally substituted with one or more substituents selected from the group consisting of halogen;
R₇ is hydrogen or C₁-C₆-alkyl;
R₈ is hydrogen or C₁-C₆-alkyl, optionally substituted with one or more substituents selected from the group consisting of halogen, cyano, NO₂ and C₁-C₄-alkoxy;
Ar₁ is aryl optionally substituted with one or more substitutents selected from the group consisting of halogen, cyano, NO₂, C₁-C₆-alkyl, C₂-C₆-alkenyl, halo-C₁-C₆-alkyl, C₁-C₆-alkoxy, C₂-C₆-alkenyloxy, halo-C₁-C₆-alkoxy, halo-C₂-C₆-alkenyloxy, C₁-C₆-alkylthio, halo-C₁-C₆-alkylthio, C₁-C₆-alkylsulfonyl, halo-C₁-C₆-alkylsulfonyl, C₁-C₆-alkylamino and di-C₁-C₆-alkylamino; and
a and b, independently of one another, are 0 or 1.

41. The compound of claim 26, wherein R₁ is aryl optionally substituted with one or more substitutents selected from the group consisting of halogen, cyano, C₁-C₄-alkyl, C₂-C₄-alkenyl, halo-C₁-C₄-alkyl, halo-C₂-C₄-alkenyl, C₁-C₄-alkoxy, C₂-C₄-alkenyloxy, halo-C₁-C₄-alkoxy, halo-C₂-C₄-alkenyloxy, C₁-C₄-alkylthio, halo-C₁-C₄-alkylthio, unsubstituted or one- to five-fold substituted aryl, unsubstituted or one- to five-fold substituted aryloxy, or unsubstituted or one- to five-fold substituted arylthio, the substituents selected from the group consisting of halogen, cyano, C₁-C₆-alkyl, halo-C₁-C₆-alkyl, C₁-C₆-alkoxy and halo-C₁-C₆-alkoxy;
R₂ is cyano;
R₃, R₄, R₅ and R₆, independently of one another, are hydrogen or C₁-C₆-alkyl;
R₇ is hydrogen or methyl;
Ar₁ is phenyl optionally substituted with one or more substitutents selected from the group consisting of halogen, cyano, NO₂, C₁-C₄-alkyl, halo-C₁-C₄-alkyl, C₁-C₄-alkoxy, halo-C₁-C₄-alkoxy, C₁-C₄-alkylthio and halo-C₁-C₄-alkylthio;
a is 1 and b is 0.

42. The compound of claim 26, wherein R₁ is phenyl optionally substituted with one or more substitutents selected from the group consisting of halogen, cyano, C₁-C₂-alkyl, halo-C₁-C₂-alkyl, C₁-C₂-alkoxy, halo-C₁-C₂-alkoxy, C₁-C₂-alkylthio, halo-C₁-C₂-alkylthio, unsubstituted or one- to five-fold substituted phenoxy, or unsubstituted or one- to five-fold substituted arylthio, the substituents selected from the group consisting of halogen, cyano, C₁-C₄-alkyl, halo-C₁-C₄-alkyl, C₁-C₄-alkoxy and halo-C₁-C₄-alkoxy;
R₂ is cyano;
R₃, R₄, R₅, R₆ and R₇ are hydrogen;
Ar₁ is phenyl optionally substituted with one or more substitutents selected from the group consisting of halogen, cyano, NO₂, C₁-C₂-alkyl, halo-C₁-C₂-alkyl, C₁-C₂-alkoxy, halo-C₁-C₂-alkoxy, C₁-C₂-alkylthio and halo-C₁-C₂-alkylthio;
X is oxygen;
Y is a direct bond;
a is 1 and b is 0.

43. The compound of claim 26, wherein R₁ is phenyl optionally substituted with one or more substitutents selected from the group consisting of halogen, cyano, methyl, halo-methyl, methoxy or unsubstituted or mono- or dihalogen substituted phenoxy;
R₂ is cyano;
R₃, R₄, R₅, R₆ and R₇ are hydrogen;
Ar₁ is phenyl optionally substituted with one or more substitutents selected from the group consisting of halogen, cyano, NO₂, methyl, halo-methyl, methoxy, halo-methoxy, methylthio and halo-methylthio;
X is oxygen;
Y is a direct bond;
a is I and b is 0.

44. The compound of claim 26, selected from the group consisting of
N-[2-(2-Chloro-phenoxy)-1,1-dicyanoethyl]-4-trifluoromethoxy-benzamide;
N-[1,1-Dicyano-2-(2-methoxy-phenoxy)-ethyl]-4-trifluoromethoxy-benzamide; and
N-{1,1-Dicyano-2-[5-cyano-2-(2,4-di-chlorophenoxy)-phenoxy]-ethyl}-4-trifluoromethoxy-benzamide.

45. A process for the preparation of a compound of formula I, 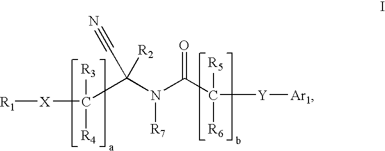
wherein,
R₁ is hydrogen, C₁-C₆-alkyl, aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉, COOR₈, unsubstituted or one- to five-fold substituted aryl, unsubstituted or one- to five-fold substituted aryloxy, unsubstituted or one- to five-fold substituted arylthio, unsubstituted or one- to five-fold substituted hetaryl or unsubstituted or one- to five-fold substituted hetaryloxy, the substituents selected from the group consisting of halogen, cyano, NO₂, hydroxy, C₁-C₆-alkyl, halo-C₁-C₆-alkyl, C₁-C₆-alkoxy, halo-C₁-C₆-alkoxy, NH₂, C₁-C₆-alkylamino, di-C₁-C₆-alkylamino, C₁-C₆-alkylthio, COR₈, CONR₈R₉ and COOR₈;
R₂ is cyano, CONR₈R₉ or COOR₈;
R₃, R₄, R₅ and R₆, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
R₇ is hydrogen, C₁-C₆-alkyl, C₂-C₆-alkenyl, C₂-C₆-alkynyl or C₃-C₆-cycloalkyl each optionally substituted with one or more substituents selected from the group consisting of halogen, cyano, hydroxy and C₁-C₆-alkoxy; or C₁-C₆-alkylcarbonyl or C₁-C₆-alkoxycarbonyl;
R₈ and R₉, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
or, R₈ and R₉, together with the nitrogen atom to which they are attached, form a ring of 3 to 6 atoms, optionally including one additional atom of nitrogen, sulfur or oxygen, optionally substituted with 1 to 4 substituents selected form the group consisting of halogen, CN, NO₂, hydroxy, C₁-C₆-alkyl and C₁-C₆-alkoxy;
Ar₁ is aryl or hetaryl each optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉ and COOR₈;
X and Y are, independently of one another, a direct bond or oxygen; and
a and b, independently of one another, are 0, 1 or 2; and
enantiomers and/or salts thereof,
said method comprising,
reacting a compound of formula II, 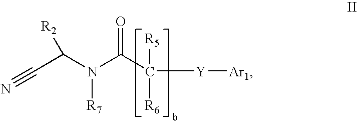
wherein, R₂, R₅, R₆, R₇, Ar₁, Y and b are as defined above for formula I, with a compound of formula III, 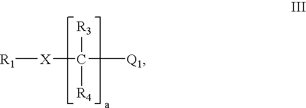
wherein, R₁, R₃, R₄, X and a are as defined for formula I, and Q₁ is a leaving group, optionally in the presence of a basic catalyst;
wherein a compound of formula I is prepared.

46. A composition for the control of parasites, comprising at least one compound of formula I, 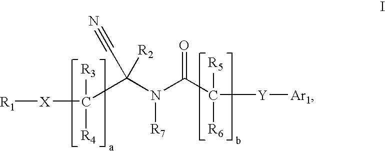
wherein,
R₁ is hydrogen, C₁-C₆-alkyl, aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉, COOR₈, unsubstituted or one- to five-fold substituted aryl, unsubstituted or one- to five-fold substituted aryloxy, unsubstituted or one- to five-fold substituted arylthio, unsubstituted or one- to five-fold substituted hetaryl or unsubstituted or one- to five-fold substituted hetaryloxy, the substituents selected from the group consisting of halogen, cyano, NO₂, hydroxy, C₁-C₆-alkyl, halo-C₁-C₆-alkyl, C₁-C₆-alkoxy, halo-C₁-C₆-alkoxy, NH₂, C₁-C₆-alkylamino, di-C₁-C₆-alkylamino, C₁-C₆-alkylthio, COR₈, CONR₈R₉ and COOR₈;
R₂ is cyano, CONR₈R₉ or COOR₈;
R₃, R₄, R₅ and R₆, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
R₇ is hydrogen, C₁-C₆-alkyl, C₂-C₆-alkenyl, C₂-C₆-alkynyl or C₃-C₆-cycloalkyl each optionally substituted with one or more substituents selected from the group consisting of halogen, cyano, hydroxy and C₁-C₆-alkoxy; or C₁-C₆-alkylcarbonyl or C₁-C₆-alkoxycarbonyl;
R₈ and R₉, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
or, R₈ and R₉, together with the nitrogen atom to which they are attached, form a ring of 3 to 6 atoms, optionally including one additional atom of nitrogen, sulfur or oxygen, optionally substituted with 1 to 4 substituents selected form the group consisting of halogen, CN, NO₂, hydroxy, C₁-C₆-alkyl and C₁-C₆-alkoxy;
Ar₁ is aryl or hetaryl each optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆₋cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉ and COOR₈;
X and Y are, independently of one another, a direct bond or oxygen; and
a and b, independently of one another, are 0, 1 or 2; and
enantiomers and/or salts thereof;
and one or more carriers and/or dispersants.

47. A process for the control of parasites comprising,
employing against said parasites an effective amount of at least one compound of formula I, 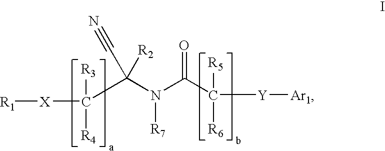
wherein,
R₁ is hydrogen, C₁-C₆-alkyl, aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉, COOR₈, unsubstituted or one- to five-fold substituted aryl, unsubstituted or one- to five-fold substituted aryloxy, unsubstituted or one- to five-fold substituted arylthio, unsubstituted or one- to five-fold substituted hetaryl or unsubstituted or one- to five-fold substituted hetaryloxy, the substituents selected from the group consisting of halogen, cyano, NO₂, hydroxy, C₁-C₆-alkyl, halo-C₁-C₆-alkyl, C₁-C₆-alkoxy, halo-C₁-C₆-alkoxy, NH₂, C₁-C₆-alkylamino, di-C₁-C₆-alkylamino, C₁-C₆-alkylthio, COR₈, CONR₈R₉ and COOR₈;
R₂ is cyano, CONR₈R₉ or COOR₈;
R₃, R₄, R₅ and R₆, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
R₇ is hydrogen, C₁-C₆-alkyl, C₂-C₆-alkenyl, C₂-C₆-alkynyl or C₃-C₆-cycloalkyl each optionally substituted with one or more substituents selected from the group consisting of halogen, cyano, hydroxy and C₁-C₆-alkoxy; or C₁-C₆-alkylcarbonyl or C₁-C₆-alkoxycarbonyl;
R₈ and R₉, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
or, R₈ and R₉, together with the nitrogen atom to which they are attached, form a ring of 3 to 6 atoms, optionally including one additional atom of nitrogen, sulfur or oxygen, optionally substituted with 1 to 4 substituents selected form the group consisting of halogen, CN, NO₂, hydroxy, C₁-C₆-alkyl and C₁-C₆-alkoxy;
Ar₁ is aryl or hetaryl each optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉ and COOR₈;
X and Y are, independently of one another, a direct bond or oxygen; and
a and b, independently of one another, are 0, 1 or 2; and
enantiomers and/or salts thereof;
wherein said parasites are controlled.

48. The process of claim 47, wherein said control of parasites is of parasites in and on warm-blooded animals.

49. A process for preparing a pharmaceutical composition against parasites in and on warm-blooded animals, said method comprising,
contacting a compound of formula I, 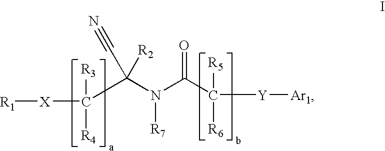
wherein,
R₁ is hydrogen, C₁-C₆-alkyl, aryl optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉, COOR₈, unsubstituted or one- to five-fold substituted aryl, unsubstituted or one- to five-fold substituted aryloxy, unsubstituted or one- to five-fold substituted arylthio, unsubstituted or one- to five-fold substituted hetaryl or unsubstituted or one- to five-fold substituted hetaryloxy, the substituents selected from the group consisting of halogen, cyano, NO₂, hydroxy, C₁-C₆-alkyl, halo-C₁-C₆-alkyl, C₁-C₆-alkoxy, halo-C₁-C₆-alkoxy, NH₂, C₁-C₆-alkylamino, di-C₁-C₆-alkylamino, C₁-C₆-alkylthio, COR₈, CONR₈R₉ and COOR₈;
R₂ is cyano, CONR₈R₉ or COOR₈;
R₃, R₄, R₅ and R₆, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
R₇ is hydrogen, C₁-C₆-alkyl, C₂-C₆-alkenyl, C₂-C₆-alkynyl or C₃-C₆-cycloalkyl each optionally substituted with one or more substituents selected from the group consisting of halogen, cyano, hydroxy and C₁-C₆-alkoxy; or C₁-C₆-alkylcarbonyl or C₁-C₆-alkoxycarbonyl;
R₈ and R₉, independently of one another, are hydrogen; C₁-C₆-alkyl, C₂-C₆-alkenyl, C₂-C₆-alkynyl, or C₃-C₆-cycloalkyl each optionally substituted with one or more substituents selected from the group consisting of halogen, cyano, NO₂ and C₁-C₆-alkoxy;
or, R₈ and R₉, together with the nitrogen atom to which they are attached, form a ring of 3 to 6 atoms, optionally including one additional atom of nitrogen, sulfur or oxygen, optionally substituted with 1 to 4 substituents selected form the group consisting of halogen, CN, NO₂, hydroxy, C₁-C₆-alkyl and C₁-C₆-alkoxy;
Ar₁ is aryl or hetaryl each optionally substituted with one or more substitutents selected from the group consisting of halogen, cyano, NO₂, C₁-C₆-alkyl, C₂-C₆-alkenyl, C₂-C₆-alkynyl, C₃-C₆-cycloalkyl, halo-C₁-C₆-alkyl, halo-C₂-C₆-alkenyl, halo-C₂-C₆-alkynyl, halo-C₃-C₆-cycloalkyl, hydroxy, C₁-C₆-alkoxy, C₂-C₆-alkenyloxy, C₂-C₆-alkynyloxy, C₃-C₆-cycloalkyloxy, halo-C₁-C₆-alkoxy, halo-C₂-C₆-alkenyloxy, halo-C₂-C₆-alkynyloxy, halo-C₃-C₆-cycloalkyloxy, SH, C₁-C₆-alkylthio, C₃-C₆-cycloalkylthio, halo-C₁-C₆-alkylthio, halo-C₃-C₆-cycloalkylthio, C₁-C₆-alkylsulfinyl, C₃-C₆-cycloalkylsulfinyl, halo-C₁-C₆-alkylsulfinyl, halo-C₃-C₆-cycloalkylsulfinyl, C₁-C₆-alkylsulfonyl, C₃-C₆-cycloalkylsulfonyl, halo-C₁-C₆-alkylsulfonyl, halo-C₃-C₆-cycloalkylsulfonyl, SO₃H, SO₂NR₈R₉, NH₂, C₁-C₆-alkylamino, C₃-C₆-cycloalkylamino, di-C₁-C₆-alkylamino, tri-C₁-C₄-alkylsilyl, COR₈, CONR₈R₉ and COOR₈;
X and Y are, independently of one another, a direct bond or oxygen; and
a and b, independently of one another, are 0, 1 or 2; and
enantiomers and/or salts thereof,
with a physiologically acceptable additive;
wherein said pharmaceutical composition is prepared.
